(19) United States
(12) Patent Application Publication
(10) Pub. No.: US 2026/0364049 A1
(43) Pub. Date: Nov. 19, 2026

(54) RRX-001 FOR THE TREATMENT OF A HEMOGLOBINOPATHY

(71) Applicant: EpicentRx, Inc., La Jolla, CA (US)

(72) Inventors: Bryan T. ORONSKY, Los Altos Hills, CA (US); Scott CAROEN, San Francisco, CA (US); Tony R. REID, San Diego, CA (US)

(21) Appl. No.: 19/136,778

(22) PCT Filed: Dec. 8, 2023
(86) PCT No.: PCT/US2023/083149

§ 371 Date: Jun. 6, 2025

Related U.S. Application Data
(60) Provisional application No. 63/431,200, filed on Dec. 8, 2022.

(51) Int. Cl. A61K 31/397; A61K 9/00; A61K 33/00; A61K 45/06; A61P 7/06

(52) U.S. Cl. CPC A61K 31/397; A61K 9/0019; A61K 33/00; A61K 45/06; A61P 7/06

(57) ABSTRACT

Methods and compositions for the treatment of a hemoglobinopathy and to reduce or prevent vasoconstriction in a subject are described. A method for treating the hemoglobinopathy and to reduce or prevent the vasoconstriction in the subject comprises administering a therapeutically effective amount of RRx-001, or a pharmaceutically acceptable salt thereof, to the subject. The therapeutically effective amount of the RRx-001, or the pharmaceutically acceptable salt thereof, may be administered to the subject in a pharmaceutical composition that also comprises a blood product and/or hydroxyurea and/or a nitric oxide-releasing or prolonging compound.

### CROSS REFERENCE TO RELATED APPLICATIONS SECTION

This application claims priority to U.S. Provisional Patent Application No. 63/431,200, filed Dec. 8, 2022, the entire contents of which are hereby incorporated by reference in its entirety.

### FIELD OF THE INVENTION

This disclosure relates to compositions and methods for the treatment of a hemoglobinopathy that is characterized by a reduced affinity of hemoglobin for oxygen and vasoconstriction for ameliorating symptoms and adverse outcomes of the hemoglobinopathy and the vasoconstriction. The treatment method is applicable for both long-term and short-term use and may be administered prophylactically and/or therapeutically in emergency, acute crisis clinical situations.

### BACKGROUND

Hemoglobinopathies are associated with a decreased affinity of oxygen for hemoglobin. Examples of hemoglobinopathies include: sickle cell disease and sickle cell trait, thalassemias, and hemoglobin variants, such as Hb-C, Hb-D, Hb-E, Hb-H, Hb-I, Hb-O-Arab, and Hb-Kansas disorders. Perhaps the most extensively studied hemoglobinopathy is sickle cell disease, which results from a mutation at the sixth amino acid from the N-terminus of the β-polypeptide chain of hemoglobin (Hb-S). Approximately 300,000 new cases of sickle cell disease are seen each year and millions of patients are affected globally. See D. Darbari, et al. (2020) Eur. J. Haematol. 105 (3): 237-346. Sickle cell anemia is an autosomal recessive blood disease to which primarily individuals of African, Mediterranean and Middle Eastern origin or their descendants are predisposed. The anemia, which is hemolytic, results from the physical aggregation of a mutant hemoglobin protein in red blood cells. This aggregation results in morphologic deformation of deoxygenated red blood cells, such that blood flow through the capillaries is impaired, which leads to morbidities, such as painful vaso-occlusive crisis (VOC) and ischemic injury. The pathophysiology of VOCs includes polymerization of abnormal sickle hemoglobin, inflammation, and adhesion. See D. Darbari, et al. (2020) Eur. J. Haematol. 105 (3): 237-346. Oxidative stress, which plays a significant role in the pathophysiology of sickle cell anemia, leads to acute inflammation, endothelial dysfunction, and progressive organ dysfunction. The mechanisms, which contribute to increased oxidative stress, include an excessive level of cell-free hemoglobin, recurrent ischemia-reperfusion injury, and iron overload. See C. Antwi-Boasiako, et al. (2019) Med. Sci. (Basel). 7 (2): 17.

Free iron and heme, which increase oxidative stress in part through excess hydroxyl radicals produced from the Fenton reaction (Fe²⁺+H₂O₂→Fe³⁺+OH·+OH⁻) damages biomolecules, lipids, proteins and DNA. Sources of excess iron and heme accumulation in sickle cell disease include increased red blood cell breakdown and lysis and ineffective red blood cell production (erythropoiesis), which lead to the release of free iron and heme, and intravenous iron supplementation and blood transfusions for the anemia that is invariably present. Scavenging or detoxification of excess free radicals is accomplished by upregulation of antioxidant or antioxidant-related genes, such as heme oxygenase (HMOX), glutamate-cysteine ligase (GCL), glutathione transferase alpha, NAD (P) dehydrogenase, and glutathione S-transferases. In addition, specific protective mechanisms to counteract the toxicity of hemoglobin and iron include the proteins haptoglobin (Hp), hemopexin (Hx), ferritin, and ferroportin.

Another related issue in sickle cell disease is reduced bioavailability of nitric oxide, due to scavenging by cell-free plasma hemoglobin. The reduced nitric oxide leads to vasoconstriction, which, in turn, exacerbates vaso-occlusion and resulting pain crises, thereby worsening the severity of sickle cell disease, and increasing the risk of damaging critical organs.

Hemoglobin molecules are comprised of two alpha polypeptide chains and two beta polypeptide chains where the sickle cell mutation, a single amino acid substitution from glutamine to valine, is present. In general, due to the differences between cells containing hemoglobin A, normal hemoglobin, and hemoglobin S, the sickle cell hemoglobin, the latter under deoxygenated conditions, form polymers in the deoxygenated state, leading to the characteristic elongated, rigid, sickle-like configuration. This causes red blood cells to lodge in and obstruct or occlude the small vessel capillaries. See E. Coleman, et al. (2007) Clin. Pediatr. (Phila). 46 (5): 386-91. The lifespan of the normal red blood cell is 120 days. The lifespan of the distorted sickle cell is 10-20 days, which leads to anemia. See O. Ilesanmi (2010) Hematol. Rep. 2 (1): e2.

Symptoms of sickle cell disease may include, but are not limited to, sickle cell crisis, where blood cells block the flow of blood to some parts of the body, frequent infections, and anemia. Symptoms of sickle cell crisis include pain in the extremities such as arms, legs, hands and feet, pain in the chest, pain in the lower back or chest, headache, tiredness, jaundice, painful erections in males, and difficulty breathing. Further symptoms of sickle cell disease may include stroke, delayed growth or puberty, swelling in the hands and feet, and vision problems.

The only approved treatment for sickle cell disease in the United States is hydroxyurea—a highly toxic, mutagenic, myelosuppressive agent that inhibits DNA synthesis. The adverse effects of hydroxyurea that have been reported include moderate drowsiness, anorexia, nausea, vomiting, diarrhea, constipation, rash, skin ulceration, dermatomyositis-like skin, skin cancer, peripheral erythema, squamous dysplasia, nail changes, facial erythema, neutropenia, interstitial pneumonia, hepatitis, and rarely renal toxicity. See R. Agrawal, et al. (2014) Indian J. Hematol. Blood Transfus. 30 (2): 91-96; and S. Lanzkron, et al. (2008) Ann. Intern. Med. 148:939-955. A wide variety of malignancies have been reported in both children and adults with sickle cell disease. It is suspected that long-term exposure to hydroxyurea, as a mutagenic agent, may increase this risk. See S. Regan, et al. (2019) Cancer Biol. Ther. 20 (11): 1389-1397.

Further, chronic pain in sickle cell disease, or pain that is present on most days and has lasted at least 6 months, can begin as early as childhood and its prevalence increases with age. See C. Dampier, et al. (2017) J. Pain 18 (5): 490-498. By adulthood, more than 55% of patients experience pain on more than half of days, with nearly one third (29%) reporting pain on 95% of days. See W. R. Smith, et al. (2008) Ann Intern Med. 2008; 148 (2): 94-101. Chronic pain associated with sickle cell disease is difficult to distinguish from the VOCs. See C. Dampier, et al. (2017) J. Pain 18 (5): 490-498. Further, acute and chronic pain in sickle cell disease is typically treated with opioids or chronic opioid therapy; however, opioid use may lead to abuse and misuse. See S. Ballas (2021) J. Clin. Med. 10 (3): 439; and I. Osunkwo, et al. (2020) Hematology Am Soc Hematol Educ Program 2020 (1): 562-569.

Therefore, a need exists for a treatment that minimizes the adverse effects of sickle cell anemia by directly inhibiting the underlying cause of sickle cell crises. A need also exists for a treatment that reduces opioid use for those suffering from sickle cell disease.

### SUMMARY

An embodiment of the disclosed subject matter includes a method for treating a hemoglobinopathy and to reduce or prevent vasoconstriction in a subject, where the method includes administering to the subject in need thereof an effective amount of RRx-001, or a pharmaceutically acceptable salt thereof. RRx-001 is a small molecule in a Phase 3 clinical trial with antioxidant and anti-inflammatory properties that donates nitric oxide only under low oxygen or hypoxic conditions.

The effective amount of RRx-001, or the pharmaceutically acceptable salt thereof, is a therapeutically effective amount of the RRx-001, or the pharmaceutically acceptable salt thereof. In some embodiments, the therapeutically effective amount of the RRx-001, or the pharmaceutically acceptable salt thereof, is in a range of about 0.1 mg and about 500.0 mg. In other embodiments, the therapeutically effective amount of the RRx-001, or the pharmaceutically acceptable salt thereof, is in a range of about 0.5 mg and about 200.0 mg.

The therapeutically effective amount of the RRx-001, or the pharmaceutically acceptable salt thereof, results in an increase in at least one of the following cytoprotectants: heme oxygenase (HMOX), ferritin heavy chain, haptoglobin (Hp), hemopexin, ferroportin, glutamate-cysteine ligase (GCL), glutathione transferase alpha, NAD (P) dehydrogenase, and/or glutathione S-transferase n1 in the subject. In some embodiments, the therapeutically effective amount of the RRx-001, or the pharmaceutically acceptable salt thereof, results in an increase in at least one of: a concentration of ferritin, nitric oxide, capillary density, oxygen delivery, oxygen uptake, a quantity of flowing venules, and/or functional capillary density (FCD) in the subject. Further, the therapeutically effective amount of the RRx-001, or the pharmaceutically acceptable salt thereof, results in a decrease in at least one of: a concentration of bilirubin, a heart rate, and/or a lactate concentration in the subject.

Additionally, the therapeutically effective amount of the RRx-001, or the pharmaceutically acceptable salt thereof, results in an amelioration of at least one adverse event of the hemoglobinopathy in the subject. In some embodiments, each adverse event of the at least one adverse event is a vaso-occlusive crisis (VOC). Each adverse event of the at least one adverse event in the subject is selected from the group consisting of: anemia, pain or swelling in at least one extremity, chest pain, lower back pain, headache, tiredness, jaundice, difficulty breathing, delayed growth or puberty, a painful erection in a male subject, and vision issues.

Further, the administering the therapeutically effective amount of the RRx-001, or the pharmaceutically acceptable salt thereof, results in an amelioration of pain associated with the hemoglobinopathy in the subject such that a reduced amount of a pain-relieving agent (such as an opioid) is administered to the subject. In some embodiments, the pain-relieving agent is a nonsteroidal anti-inflammatory agent (NSAID). In some embodiments, the pain-relieving agent is the opioid. In some embodiments, the pain-relieving agent is acetaminophen. In some embodiments, the pain-relieving agent is the anesthetic. In some embodiments, the pain-relieving agent is the sodium channel blocker. In some embodiments, the pain-relieving agent is the gabapentinoid (e.g., Neurontin or Gabapentin). In some embodiments, the pain-relieving agent is tramadol. In some embodiments, the pain-relieving agent is tapentadol. In some embodiments, the pain-relieving agent is the cannabinoid. In some embodiments, the pain-relieving agent is Ziconotide.

In some embodiments, prior to administering the effective amount of RRx-001, or the pharmaceutically acceptable salt thereof, the method comprises diagnosing the subject as suffering from the hemoglobinopathy or identifying the subject as being at risk for incurring the hemoglobinopathy. In some embodiments, prior to administering the effective amount of RRx-001, or the pharmaceutically acceptable salt thereof, the method comprises diagnosing the subject as suffering from a sickle cell crisis or identifying the subject as being at risk for incurring the sickle cell crisis.

In some embodiments, administration of the effective amount of RRx-001, or the pharmaceutically acceptable salt thereof, occurs via a single administration. In other embodiments, administration of the effective amount of RRx-001, or the pharmaceutically acceptable salt thereof, occurs via at least two administrations. In some embodiments, administration of the effective amount of RRx-001, or the pharmaceutically acceptable salt thereof, occurs at a frequency in a range of about 1 time per hour and about 1 time per month. In some embodiments, administration of the effective amount of RRx-001, or the pharmaceutically acceptable salt thereof, occurs at a frequency in a range of about 4 times per day and about 1 time per week. In some embodiments, administration of the effective amount of RRx-001, or the pharmaceutically acceptable salt thereof, occurs at a frequency in a range of about 2 times per day and about 3 times per week. In some embodiments, administration of the effective amount of RRx-001, or the pharmaceutically acceptable salt thereof, occurs at a frequency in a range of about one time a day. In some embodiments, administration of the effective amount of RRx-001, or the pharmaceutically acceptable salt thereof, occurs at a frequency in a range of about 1 time per day and about 2 times per week.

In some embodiments, the subject is a mammal subject. In other embodiments, the mammal subject is a human subject. In other embodiments, the mammal subject is a non-human subject. In some embodiments, administering the therapeutically effective amount of RRx-001, or a pharmaceutically acceptable salt thereof, occurs via oral administration, transdermal administration, administration by inhalation, nasal administration, topical administration, intra-aural administration, rectal administration, intravenous administration, intramuscular administration, subcutaneous administration, or intraperitoneal administration, or combinations thereof. In some embodiments, administering the effective amount of RRx-001, or the pharmaceutically acceptable salt thereof, occurs via parenteral administration. In some embodiments, administering the therapeutically effective amount of RRx-001, or a pharmaceutically acceptable salt thereof, occurs via an oral administration. In some embodiments, administering the therapeutically effective amount of RRx-001, or a pharmaceutically acceptable salt thereof, occurs percutaneously. In some embodiments, administering the therapeutically effective amount of RRx-001, or a pharmaceutically acceptable salt thereof, occurs epicardially. In some embodiments, administering the therapeutically effective amount of RRx-001, or a pharmaceutically acceptable salt thereof, occurs endocardially. In some embodiments, administering the therapeutically effective amount of RRx-001, or a pharmaceutically acceptable salt thereof, occurs via an intravenous administration. In some embodiments, administering the therapeutically effective amount of RRx-001, or a pharmaceutically acceptable salt thereof, occurs via a direct intratumoral injection. In some embodiments, administering the effective amount of RRx-001, or the pharmaceutically acceptable salt thereof, occurs via intravenous administration, an oral administration, a subcutaneous administration, or an intramuscular administration.

In some embodiments, the method also includes administering an agent (e.g., a nitric oxide-releasing or prolonging compound) before, during, or after the administration of the effective amount of RRx-001, or the pharmaceutically acceptable salt thereof, where the nitric oxide-releasing or prolonging compound is a phosphodiesterase inhibitor, inhaled nitric oxide, inhaled carbon monoxide, and/or nitrite.

Another embodiment of disclosed subject matter includes a method for treating sickle cell disease in a subject, where the method includes administering to the subject in need thereof a composition comprising the therapeutically effective amount of the RRx-001, or the pharmaceutically acceptable salt thereof, and a blood product. In some embodiments, the composition further comprises hydroxyurea. In other embodiments, the blood product comprises erythrocyte cells. In some embodiments, the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis. In other embodiments, the blood product is a mixture of packed red blood cells. In some embodiments, the blood product is whole blood. In some embodiments, the whole blood is autologous whole blood. In some embodiments, the whole blood is donor-matched allogenic whole blood.

A further embodiment of the disclosed subject matter includes the pharmaceutical composition for treating sickle cell disease in a subject in need thereof. The pharmaceutical composition comprises the effective amount of RRx-001, or the pharmaceutically acceptable salt thereof. In some embodiments, the pharmaceutical composition further comprises the blood product. In some embodiments, the pharmaceutical composition further comprises hydroxyurea.

Various enumerated embodiments are contemplated herein. For example, in Embodiment 1, a method for treating a hemoglobinopathy and to reduce or prevent vasoconstriction in a subject is described. The method comprises administering to the subject in need thereof an effective amount of RRx-001, or a pharmaceutically acceptable salt thereof.

Embodiment 2. The method of Embodiment 1, wherein the effective amount of RRx-001, or the pharmaceutically acceptable salt thereof, is a therapeutically effective amount of the RRx-001, or the pharmaceutically acceptable salt thereof.

Embodiment 3. The method of Embodiment 2, wherein the therapeutically effective amount of the RRx-001, or the pharmaceutically acceptable salt thereof, is in a range of about 0.1 mg and about 500.0 mg.

Embodiment 4. The method of Embodiment 3, wherein the therapeutically effective amount of the RRx-001, or the pharmaceutically acceptable salt thereof, is in a range of about 0.5 mg and about 200.0 mg.

Embodiment 5. The method of any of Embodiments 2-4, wherein the therapeutically effective amount of the RRx-001, or the pharmaceutically acceptable salt thereof, results in an increase in at least one of: nitric oxide (NO), heme oxygenase (HMOX), ferritin heavy chain, haptoglobin (Hp), hemopexin, ferroportin, glutamate-cysteine ligase (GCL), glutathione transferase alpha, NAD (P) dehydrogenase, glutathione S-transferase n1, a concentration of ferritin, capillary density, oxygen delivery, oxygen uptake, a quantity of flowing venules, and functional capillary density in the subject.

Embodiment 6. The method of any of Embodiments 2-5, wherein the therapeutically effective amount of the RRx-001, or the pharmaceutically acceptable salt thereof, results in a decrease in at least one of: a concentration of bilirubin, a heart rate, and a lactate concentration in the subject.

Embodiment 7. The method of any of Embodiments 2-6, wherein the therapeutically effective amount of the RRx-001, or the pharmaceutically acceptable salt thereof, results in an amelioration of at least one adverse event of the hemoglobinopathy in the subject.

Embodiment 8. The method of Embodiment 7, wherein each adverse event of the at least one adverse event is a vaso-occlusive crisis (VOC).

Embodiment 9. The method of Embodiment 7, wherein each adverse event of the at least one adverse event is selected from the group consisting of: anemia, pain or swelling in at least one extremity, chest pain, lower back pain, headache, tiredness, jaundice, difficulty breathing, delayed growth or puberty, painful erection in a male subject, and vision issues.

Embodiment 10. The method of any of Embodiments 2-6, wherein the therapeutically effective amount of the RRx-001, or the pharmaceutically acceptable salt thereof, results in an amelioration of pain associated with the hemoglobinopathy in the subject such that a reduced amount of a pain-relieving agent is administered to the subject.

Embodiment 11. The method of any of Embodiments 1-10, wherein, prior to administering the effective amount of RRx-001, or the pharmaceutically acceptable salt thereof, diagnosing the subject as suffering from a sickle cell crisis.

Embodiment 12. The method of any of Embodiments 1-10, wherein, prior to administering the effective amount of RRx-001, or the pharmaceutically acceptable salt thereof, identifying the subject as being at risk for incurring a sickle cell crisis.

Embodiment 13. The method of any of Embodiments 1-12, wherein administering the effective amount of RRx-001, or the pharmaceutically acceptable salt thereof, is performed via a single administration.

Embodiment 14. The method of any of Embodiments 1-12, wherein administering the effective amount of RRx-001, or the pharmaceutically acceptable salt thereof, is performed via at least two administrations.

Embodiment 15. The method of Embodiment 14, wherein administering the effective amount of RRx-001, or the pharmaceutically acceptable salt thereof, is performed a frequency in a range of about 1 time per hour and about 1 time per month.

Embodiment 16. The method of any of Embodiments 1-15, wherein the subject is a mammal subject.

Embodiment 17. The method of Embodiment 16, wherein the mammal subject is a human subject.

Embodiment 18. The method of any of Embodiments 1-17, wherein administering the effective amount of RRx-001, or the pharmaceutically acceptable salt thereof, occurs via parenteral administration.

Embodiment 19. The method of any of Embodiments 1-17, wherein administering the effective amount of RRx-001, or the pharmaceutically acceptable salt thereof, occurs via an intravenous administration, an oral administration, a subcutaneous administration, or an intramuscular administration.

Embodiment 20. The method of any of Embodiments 1-19, further comprising: administering a nitric oxide-releasing or prolonging compound before, during, or after the administration of the effective amount of RRx-001, or the pharmaceutically acceptable salt thereof.

Embodiment 21. The method of Embodiment 20, wherein the nitric oxide-releasing or prolonging compound is selected from the group consisting of: a phosphodiesterase inhibitor, inhaled nitric oxide, inhaled carbon monoxide, and nitrite.

Embodiment 22. The method of any of Embodiments 2-21, wherein the therapeutically effective amount of the RRx-001, or the pharmaceutically acceptable salt thereof, is administered as a composition comprising a blood product.

Embodiment 23. The method of Embodiment 22, wherein the therapeutically effective amount of the RRx-001, or the pharmaceutically acceptable salt thereof, is in a range of about 0.1 mg and about 500.0 mg.

Embodiment 24. The method of Embodiment 23, wherein the therapeutically effective amount of the RRx-001, or the pharmaceutically acceptable salt thereof, is in a range of about 0.5 mg and about 200.0 mg.

Embodiment 25. The method of any of Embodiments 22-24, wherein administering the composition results in an increase in at least one of: nitric oxide (NO), heme oxygenase (HMOX), ferritin heavy chain, haptoglobin (Hp), hemopexin, ferroportin, glutamate-cysteine ligase (GCL), glutathione transferase alpha, NAD (P) dehydrogenase, glutathione S-transferase η1, a concentration of ferritin, capillary density, oxygen delivery, oxygen uptake, a quantity of flowing venules, and functional capillary density in the subject.

Embodiment 26. The method of any of Embodiments 22-25, wherein administering the composition results in a decrease in at least one of: a concentration of bilirubin, a heart rate, and a lactate concentration in the subject.

Embodiment 27. The method of any of Embodiments 22-26, wherein administering the composition results in an amelioration of at least one adverse event of the hemoglobinopathy in the subject.

Embodiment 28. The method of Embodiment 27, wherein each adverse event of the at least one adverse event is a vaso-occlusive crisis (VOC).

Embodiment 29. The method of Embodiment 28, wherein each adverse event of the at least one adverse event is selected from the group consisting of: anemia, pain or swelling in at least one extremity, chest pain, lower back pain, headache, tiredness, jaundice, difficulty breathing, delayed growth or puberty, painful erection in a male subject, and vision issues.

Embodiment 30. The method of any of Embodiments 22-29, wherein the therapeutically effective amount of the RRx-001, or the pharmaceutically acceptable salt thereof, results in an amelioration of pain associated with the hemoglobinopathy in the subject such that a reduced amount of a pain-relieving agent is administered to the subject.

Embodiment 31. The method of any of Embodiments 22-30, wherein, prior to administering the composition, the method further comprises: diagnosing the subject as suffering from a sickle cell crisis.

Embodiment 32. The method of any of Embodiments 22-30, wherein, prior to administering the composition, the method further comprises: identifying the subject as being at risk for incurring a sickle cell crisis.

Embodiment 33. The method of any of Embodiments 22-32, wherein administering the composition occurs via a single administration.

Embodiment 34. The method of any of Embodiments 22-32, wherein administering the composition occurs via at least two administrations.

Embodiment 35. The method of Embodiment 34, wherein administering the composition occurs at a frequency in a range of about 1 time per hour and about 1 time per month.

Embodiment 36. The method of any of Embodiments 22-35, wherein the subject is a mammal subject.

Embodiment 37. The method of Embodiment 36, wherein the mammal subject is a human subject.

Embodiment 38. The method of any of Embodiments 22-37, wherein administering the composition occurs via parenteral administration.

Embodiment 39. The method of any of Embodiments 22-37, wherein administering the effective amount of RRx-001, or the pharmaceutically acceptable salt thereof, occurs via an intravenous administration, an oral administration, a subcutaneous administration, or an intramuscular administration.

Embodiment 40. The method of any of Embodiments 22-39, wherein the composition further comprises hydroxyurea.

Embodiment 41. The method of any of Embodiments 22-40, wherein the blood product comprises erythrocyte cells.

Embodiment 42. The method of Embodiment 41, wherein the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis.

Embodiment 43. The method of any of Embodiments 22-42, wherein the blood product is a mixture of packed red blood cells.

Embodiment 44. The method of any of Embodiments 22-42, wherein the blood product is whole blood.

Embodiment 45. The method of Embodiment 44, wherein the whole blood is autologous whole blood.

Embodiment 46. The method of any of Embodiments 22-45, further comprising: administering a nitric oxide-releasing or prolonging compound before, during, or after the administration of the effective amount of RRx-001, or the pharmaceutically acceptable salt thereof.

Embodiment 47. The method of Embodiment 46, wherein the nitric oxide-releasing or prolonging compound is selected from the group consisting of: a phosphodiesterase inhibitor, inhaled nitric oxide, inhaled carbon monoxide, and nitrite.

Embodiment 48. A composition for treating a hemoglobinopathy and to reduce or prevent vasoconstriction in a subject in need thereof, the composition comprising an effective amount of RRx-001, or a pharmaceutically acceptable salt thereof.

Embodiment 49. The composition of Embodiment 48, further comprising a blood product.

Embodiment 50. The composition of Embodiment 49, wherein the blood product comprises erythrocyte cells.

Embodiment 51. The composition of Embodiment 50, wherein the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis.

Embodiment 52. The composition of Embodiment 49, wherein the blood product is a mixture of packed red blood cells.

Embodiment 53. The composition of Embodiment 49, wherein the blood product is whole blood.

Embodiment 54. The composition of Embodiment 53, wherein the whole blood is autologous whole blood.

Embodiment 55. The composition of any of Embodiments 48-54, further comprising hydroxyurea.

Embodiment 56. The composition of any of Embodiments 48-55, wherein the subject suffers from a sickle cell crisis.

Embodiment 57. The composition of any of Embodiments 48-55, wherein the subject identified as being at risk for incurring a sickle cell crisis.

Embodiment 58. The composition of any of Embodiments 48-57, wherein the effective amount of RRx-001, or the pharmaceutically acceptable salt thereof, is a therapeutically effective amount of the RRx-001, or the pharmaceutically acceptable salt thereof.

Embodiment 59. The composition of Embodiment 58, wherein the therapeutically effective amount of the RRx-001, or the pharmaceutically acceptable salt thereof, is in a range of about 0.1 mg and about 500.0 mg.

Embodiment 60. The composition of Embodiment 59, wherein the therapeutically effective amount of the RRx-001, or the pharmaceutically acceptable salt thereof, is in a range of about 0.5 mg and about 200.0 mg.

Embodiment 61. The composition of any of Embodiments 48-60, wherein an administration of the composition results in an increase in at least one of: nitric oxide (NO), heme oxygenase (HMOX), ferritin heavy chain, haptoglobin (Hp), hemopexin, ferroportin, glutamate-cysteine ligase (GCL), glutathione transferase alpha, NAD (P) dehydrogenase, glutathione S-transferase n1, a concentration of ferritin, capillary density, oxygen delivery, oxygen uptake, a quantity of flowing venules, and functional capillary density in the subject.

Embodiment 62. The composition of any of Embodiments 48-61, wherein an administration of the composition results in a decrease in at least one of: a concentration of bilirubin, a heart rate, and a lactate concentration in the subject.

Embodiment 63. The composition of any of Embodiments 48-62, wherein an administration of the composition results in an amelioration of at least one adverse event of the hemoglobinopathy in the subject.

Embodiment 64. The composition of Embodiment 63, wherein each adverse event of the at least one adverse event is a vaso-occlusive crisis (VOC).

Embodiment 65. The composition of Embodiment 64, wherein each adverse event of the at least one adverse event is selected from the group consisting of: anemia, pain or swelling in at least one extremity, chest pain, lower back pain, headache, tiredness, jaundice, difficulty breathing, delayed growth or puberty, painful erection in a male subject, and vision issues.

Embodiment 66. The method of any of Embodiments 48-65, wherein the therapeutically effective amount of the RRx-001, or the pharmaceutically acceptable salt thereof, results in an amelioration of pain associated with the hemoglobinopathy in the subject such that a reduced amount of a pain-relieving agent is administered to the subject.

Embodiment 67. The composition of any of Embodiments 48-66, wherein the composition is administered via a single administration.

Embodiment 68. The composition of any of Embodiments 48-66, wherein the composition is administered via at least two administrations.

Embodiment 69. The composition of Embodiment 68, wherein the composition is administered at a frequency in a range of about 1 time per hour and about 1 time per month.

Embodiment 70. The composition of any of Embodiments 48-69, wherein the subject is a mammal subject.

Embodiment 71. The composition of Embodiment 70, wherein the mammal subject is a human subject.

Embodiment 72. The composition of any of Embodiments 48-71, wherein the composition is administered via a parenteral administration.

Embodiment 73. The composition of Embodiment 72, wherein the composition is administered via an intravenous administration, an oral administration, a subcutaneous administration, or an intramuscular administration.

### BRIEF DESCRIPTION OF THE DRA WINGS

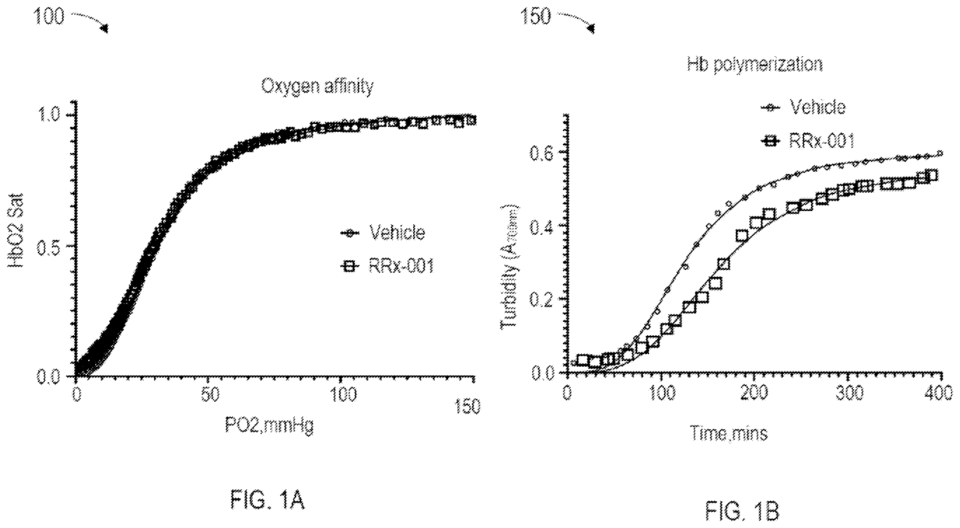

FIG. 1A is a graph of hemoglobin oxygen saturation (HbO₂ Sat) compared to oxygen pressure (PO₂, mmHg) to measure oxygen affinity, according to at least some embodiments disclosed herein.

FIG. 1B is a graph of hemoglobin polymerization as measured by turbidity (A_{700 nm}) over time, according to at least some embodiments disclosed herein.

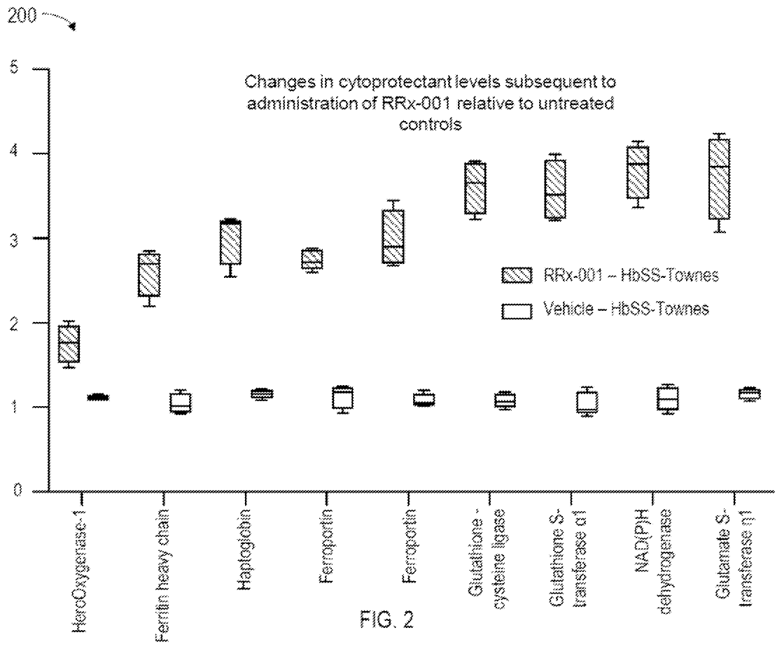

FIG. 2 is a graph showing changes in various cytoprotectant levels subsequent to administration of RRx-001, according to at least some embodiments disclosed herein.

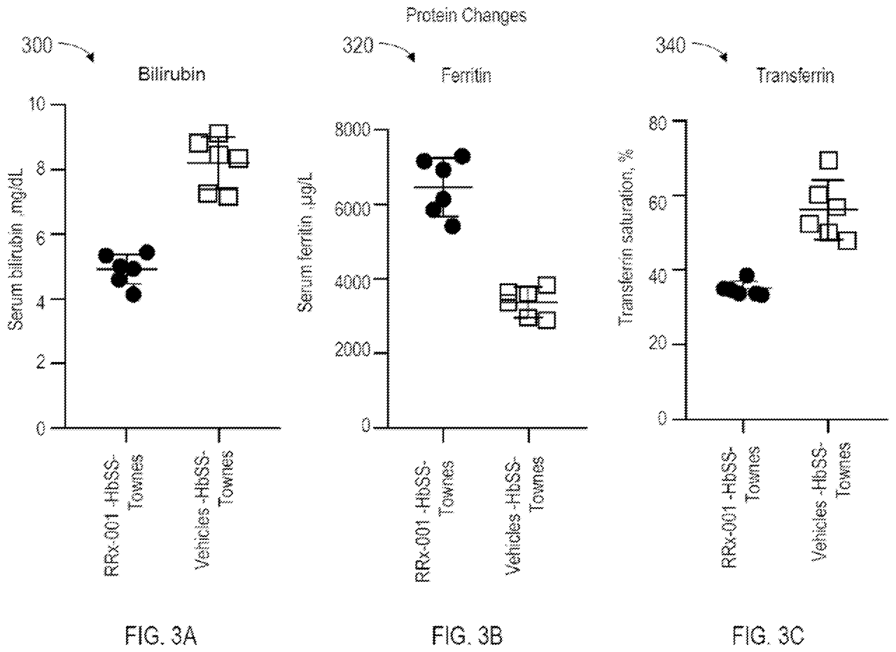

FIG. 3A is a graph showing a change in bilirubin concentration for a control and for a subject group treated with RRx-001, according to at least some embodiments disclosed herein.

FIG. 3B is a graph showing a change in ferritin concentration for a control and for a subject group treated with RRx-001, according to at least some embodiments disclosed herein.

FIG. 3C is a graph showing a change in transferrin saturation for a control and for a subject group treated with RRx-001, according to at least some embodiments disclosed herein.

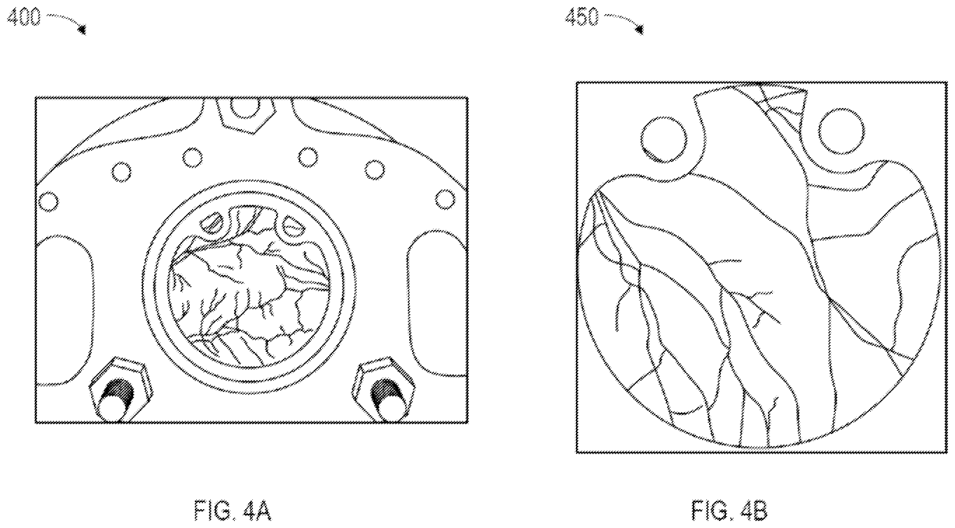

FIG. 4A is a photograph of a window chamber as it displays a vasculature of a Townes mouse, according to at least some embodiments disclosed herein.

FIG. 4B is a magnified photograph of a window chamber as it displays a vasculature of a Townes mouse, according to at least some embodiments disclosed herein.

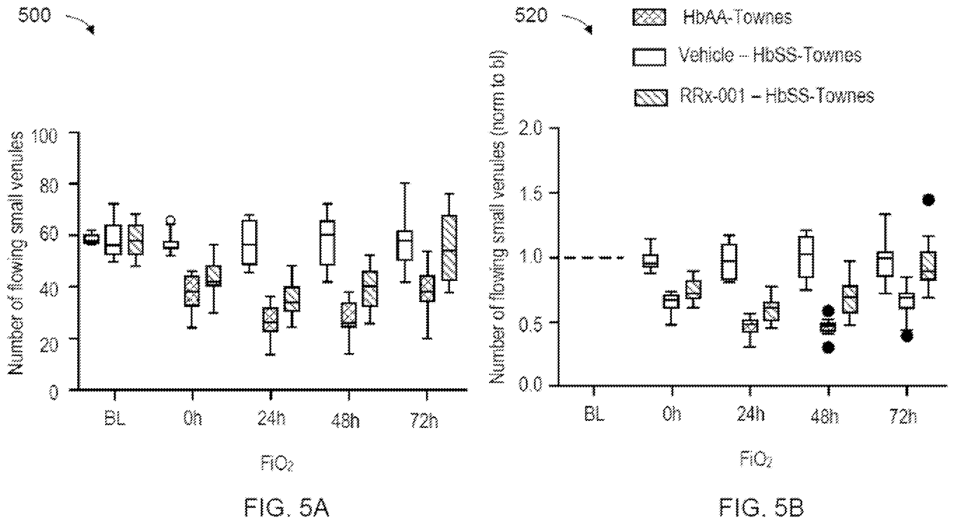

FIG. 5A is a graph showing the number of small venules in subject groups of mice over a three-day period, according to at least some embodiments disclosed herein.

FIG. 5B is a graph showing the number of small venules in subject groups of mice over a three-day period normalized to a baseline (BL) measurement, according to at least some embodiments disclosed herein.

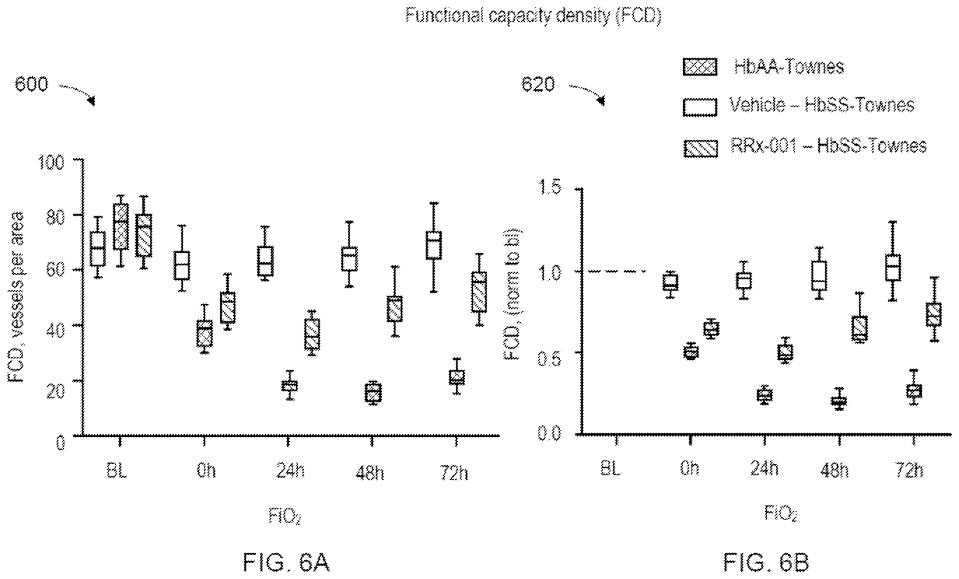

FIG. 6A is a graph of functional capillary density (FCD) in vessels per unit area in subject groups of mice over a three-day period, according to at least some embodiments disclosed herein.

FIG. 6B is a graph of functional capillary density (FCD) normalized to a baseline (BL) measurement in subject groups of mice over a three-day period, according to at least some embodiments disclosed herein.

### DETAILED DESCRIPTION

Unless specifically defined otherwise all technical and scientific terms used herein shall be taken to have the same meaning as commonly understood by one of ordinary skill in the art.

### DEFINITIONS

The terms “a” and “an” as used herein mean “one or more” and include the plural unless the context is inappropriate.

Reference to “about” a value or parameter herein includes (and describes) variations that are directed to that value or parameter per se. In embodiments, the term “about” refers to +/−10%, +/−5%, or +/−1%, of the designated value.

The “comprise” or variations such as “comprises” or “comprising,” will be understood to imply the inclusion of a stated element, integer or step, or group of elements, integers or steps, but not the exclusion of any other element, integer or step, or group of elements, integers or steps. Embodiments described herein also include “consisting” and/or “consisting essentially of” aspects.

“Treatment”, “treating” or similar phrases refer to obtaining beneficial or desired results, such as clinical results, for a subject, suffering from the hemoglobinopathy (such as sickle cell disease). As used herein, the term “treating” includes any effect, e.g., lessening, reducing, modulating, ameliorating or eliminating, that results in the improvement of the condition, disease, disorder, and the like, or ameliorating a symptom thereof. Beneficial or desired results include any one or more of: alleviating one or more symptoms of sickle cell disease, diminishing the extent of the hemoglobinopathy (such as the sickle cell disease), delaying or slowing the disease or disorder progression, and improving quality of life.

As described herein, “hemoglobinopathy” refers to a group of inherited disorders that result in abnormal production or structure of hemoglobin. Examples of hemoglobinopathies include: sickle cell disease and sickle cell trait, the thalassemias, and hemoglobin variants, such as Hb-C, Hb-D, Hb-E, Hb-H, Hb-I, Hb-O-Arab, and Hb-Kansas disorders. See Howard J. Sickle cell disease and other hemoglobinopathies. In: Goldman L, Schafer AI, eds. Goldman-Cecil Medicine. 26th ed. Philadelphia, PA: Elsevier; 2020: chap 154; and K. Smith-Whitley, et al. Hemoglobinopathies. In: Kliegman R M, St. Geme J W, Blum N J, Shah S S, Tasker R C, Wilson K M, eds. Nelson Textbook of Pediatrics. 21st ed. Philadelphia, PA: Elsevier; 2020: chap 489, the contents of which are hereby incorporated by reference in their entirety.

As described herein, “sickle cell disease” refers to a group of inherited red blood cell disorders. The most common types of sickle cell disease include HbSS, HbSC, and HbS beta thalassemia. Symptoms of sickle cell disease may include, but are not limited to, sickle cell crisis, where blood cells block the flow of blood to some parts of the body, frequent infections, and anemia. Symptoms of sickle cell crisis include pain in the extremities such as arms, legs, hands and feet, pain in the chest, pain in the lower back or chest, headache, tiredness, jaundice, painful erections in males, and difficulty breathing. Further symptoms of sickle cell disease may include stroke, delayed growth or puberty, swelling in the hands and feet, and vision problems. Sickle cell anemia is an autosomal recessive blood disease.

“Heme oxygenase” (or HMOX) is an enzyme that catalyzes the degradation of heme to produce biliverdin, ferrous ion, and carbon monoxide.

“Ferritin heavy chain” is a ferroxidase enzyme that in human subjects is encoded by the FTH1 gene. Mutation of the FTH1 gene causes Hemochromatosis type 5.

“Haptoglobin” (or Hp) is a protein that in human subjects is encoded by the HP gene. In blood plasma, Hp binds with high affinity to free hemoglobin released from erythrocytes to inhibit its harmful oxidative activity.

“Hemopexin” (or beta-1B-glycoprotein) is a glycoprotein that in human subjects is encoded by the HPX gene.

“Ferroportin-1” is a transmembrane protein that in human subjects is encoded by the SLC40A1 gene and is an iron exporter.

“Glutamate-cysteine ligase” (or GCL) synthesizes γ-glutamyl-cysteine (γ-GC) and is the rate-limiting enzyme in glutathione (or GSH) biosynthesis. The transcription factor nuclear factor erythroid 2-related factor 2 (Nrf2) regulates gene expression implicated in free radical scavenging and cytoprotection including the antioxidant glutathione (GSH) pathway. See I. Amara, et al. (2020) Cell Stress Chapeon. 25 (6): 919-928, the contents of which are hereby incorporated by reference in its entirety. Dysregulation of GCL is involved in several diseases, including diabetes, Parkinson's disease, Alzheimer's disease, COPD, HIV/AIDS, and cancer.

“Glutathione S-transferases” (or GSTs) are a family of eukaryotic and prokaryotic phase II metabolic isozymes that catalyze the conjugation of the reduced form of glutathione (GSH) to xenobiotic substrates for the purpose of detoxification.

“Ferritin” is an intracellular iron-storage protein and in human subjects that acts as a buffer against iron deficiency and iron overload. Plasma ferritin is an indirect marker of a total amount of iron stored in a subject.

A “venule” is a blood vessel in the microcirculation ranging from about 7 μm to about 1 mm in diameter that allows blood to return from the capillary beds to drain into the veins.

“Functional capillary density” (or FCD) is defined as a length of red cell-perfused capillaries per observation area (cm-1). FCD is a parameter obtained by intravital microscopy using epi-illumination of the tissue surface or trans-illumination of thin tissue layers. FCD may be used as an indicator of the quality of tissue perfusion in various animal models. See D. Nolte, et al. (1995) Int. J. Microcirc. 15:244-249, the contents of which are hereby incorporated by reference in its entirety.

“Bilirubin” is a compound that breaks down heme in vertebrates.

“Transferrins” are glycoproteins found in vertebrates that bind to and mediate the transport of iron through blood plasma.

“Lactate” is a hydroxy monocarboxylic acid anion that is the conjugate base of lactic acid.

As described herein, “vaso-occlusive crisis” (or VOC) is a form of sickle cell crisis that is believed to be caused when blood flow is blocked by sickled erythrocytes.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route.

As used herein, the term “subject” refers to an organism to be treated by the methods of the present invention. Such organisms are preferably mammals (e.g., murines, simians, equines, bovines, porcines, canines, felines, and the like), and more preferably humans. In some embodiments, the subject is a human. In some embodiments, the subject is a non-human subject.

The phrase “parenteral administration” and “administered parenterally” as used herein means modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

As used herein, the term “pharmaceutically acceptable salt” refers to any salt (e.g., acid or base) of a compound of the present invention suitable for pharmaceutical administration which, upon administration to the subject, is capable of providing a compound of this invention or an active metabolite or residue thereof. As is known to those of skill in the art, “salts” of the compounds of the present invention may be derived from inorganic or organic acids and bases.

Examples of acids include, but are not limited to, hydrochloric, hydrobromic, sulfuric, nitric, perchloric, fumaric, maleic, phosphoric, glycolic, lactic, salicylic, succinic, toluene psulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW⁴⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH⁴⁺, and NW⁴⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The terms “ABDNAZ” and “RRx-001” are used interchangeably and refer to the compound having the following structure:

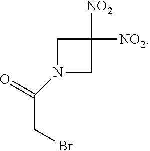 [SMILES: O=C(CBr)N1CC([N+](=O)[O-])([N+](=O)[O-])C1]

RRx-001 is associated with the chemical name 2-bromo-1-(3,3-dinitroazetidin-1-yl) ethan-1-one and is a small cyclic nitro compound that is a minimally toxic anticancer agent in Phase 3 clinical trial for the treatment of cancer. Further, RRx-001 is an electrophilic stress regulator with anti-oxidative/anti-inflammatory, vasodilatory, and cardioprotective properties. These effects are mediated by Nrf2 activation and NLRP3 inhibition, as well as nitric oxide generation under hypoxia. NLR family pyrin domain containing 3 (NLRP3) is expressed in immune cells, especially in dendritic cells and macrophages and acts as a constituent of the inflammasome. See Ghafouri-Fard (2022) Front. Immunol. 13:926895, the contents of which are hereby incorporated by reference in its entirety. NLRP3 acts as a pattern recognition receptor identifying pathogen-associated molecular patterns and also recognizes damage-associated molecular patterns. See Ghafouri-Fard (2022) Front. Immunol. 13:926895, the contents of which are hereby incorporated by reference in its entirety. Triggering of NLRP3 inflammasome by molecules ATP released from injured cells results in the activation of the inflammatory cytokines IL-1b and IL-18. Abnormal activation of NLRP3 inflammasome stimulates inflammatory or metabolic diseases. See Ghafouri-Fard (2022) Front. Immunol. 13:926895, the contents of which are hereby incorporated by reference in its entirety. As such, NLRP3 is a target for decreasing activity of NLRP3 inflammasome.

The present subject matter treats a hemoglobinopathy that is characterized by a reduced affinity of hemoglobin for oxygen and vasoconstriction for ameliorating symptoms and adverse outcomes of the hemoglobinopathy and vasoconstriction. The treatment method is applicable for both long-term and short-term use and may be administered prophylactically and/or therapeutically in emergency, acute crisis clinical situations.

Sickle cell disease results in compromised blood flow to different organs, resulting in infarction and tissue death. Another related issue in sickle cell disease is reduced bioavailability of nitric oxide, due to scavenging by cell-free plasma hemoglobin. The reduced nitric oxide leads to vasoconstriction, which, in turn, exacerbates vaso-occlusion and resulting pain crises, thereby worsening the severity of sickle cell disease, and increasing the risk of damaging critical organs. RRx-001 donates nitric oxide only under low oxygen or hypoxic conditions. Nitric oxide is a strong vasodilator. RRx-001 delivered in blood provides therapeutic “vasculoprotection,” as it not only dilates the obstructed blood vessels, resulting in improvements in blood flow, but also protects them from inflammation and oxidative damage so tissues continue to receive enough oxygen.

In some embodiments, the present method treats sickle cell anemia through the administration of a therapeutically effective dose of RRx-001 or a pharmaceutically acceptable salt thereof to the subject. The compound of this invention, RRx-001, is active in sickle cell anemia both for its ability to desickle or depolymerize the aberrant hemoglobin-S, for its antioxidant and anti-inflammatory properties, which decrease the oxidative stress and inflammation that play a key role in the pathophysiology of hemolysis, vessel occlusion, and the organ damage in sickle cell subjects and for its ability to donate nitric oxide under low oxygen or hypoxic conditions. The importance of the latter is that systemic release of nitric oxide leads to low blood pressure (hypotension) and possibly methemoglobinemia, both of which would induce the opposite effect of what is intended, namely, to increase microvascular blood flow and oxygen saturation in poorly perfused key tissues such as the heart, the brain, the lungs, the bones, and skeletal muscles. By contrast, RRx-001 only releases nitric oxide focally on demand not systemically in areas where low oxygen and vasoconstriction are present. See M. Fens, et al. (2016) Med. Oncol. 33 (8): 85, the contents of which are hereby incorporated by reference in its entirety.

Certain compounds contained in compositions of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

### RRx-001 and Sickle Cell Disease

RRx-001 may be used to treat or ameliorate various symptoms of sickle cell disease. In various embodiments, the therapeutically effective amount of the RRx-001, or the pharmaceutically acceptable salt thereof, may be used to alleviate one or more symptoms of sickle cell crisis, which include pain in the extremities such as arms, legs, hands and feet, pain in the chest, pain in the lower back or chest, headache, tiredness, jaundice, painful erections in males, and difficulty breathing. In some embodiments, the therapeutically effective amount of the RRx-001, or the pharmaceutically acceptable salt thereof, may be administered by the extracorporeal reaction of RRx-001 with the subjects own blood, either stored or fresh. In an exemplary embodiment, the RRx-001 may be administered orally.

The method of the present invention may be used to treat sickle cell disease prophylactically to decrease hemoglobin S polymerization and to prevent or reduce vasoconstriction, thereby diminishing the time and duration of red cell sickling and vaso-occlusion in the blood circulation. In addition, the methodology may be used therapeutically in patients with acute sickle cell crisis.

In various embodiments, the one or more symptoms of sickle cell disease that are ameliorated subsequent to administration of the therapeutically effective amount of the RRx-001, or the pharmaceutically acceptable salt thereof, include delayed growth or puberty, frequent infections, issues with vision, and swelling in the hands and feet. In various embodiments, administering the therapeutically effective amount of the RRx-001, or the pharmaceutically acceptable salt thereof, results in an increase in at least one of the following cytoprotectants: heme oxygenase (HMOX), ferritin heavy chain, haptoglobin (Hp), hemopexin, ferroportin, glutamate-cysteine ligase (GCL), glutathione transferase alpha, NAD (P) dehydrogenase, and/or glutathione S-transferase n1 in the subject. These levels may be measurable by various means such as via an enzyme-linked immunosorbent assay (ELISA). In some embodiments, the therapeutically effective amount of the RRx-001, or the pharmaceutically acceptable salt thereof, results in an increase in at least one of: a concentration of ferritin, nitric oxide, capillary density, oxygen delivery, oxygen uptake, a quantity of flowing venules, and/or functional capillary density (FCD) in the subject. Further, the therapeutically effective amount of the RRx-001, or the pharmaceutically acceptable salt thereof, results in a decrease in at least one of: a concentration of bilirubin, a heart rate, and/or a lactate concentration in the subject.

In other embodiments, administering the therapeutically effective amount of the RRx-001, or the pharmaceutically acceptable salt thereof, results in an amelioration of at least one adverse event of the hemoglobinopathy. For instance, administering the therapeutically effective amount of the RRx-001, or the pharmaceutically acceptable salt thereof, ameliorates the VOC. Each adverse event of the at least one adverse event in the subject is selected from the group consisting of: anemia, pain or swelling in at least one extremity, chest pain, lower back pain, headache, tiredness, jaundice, difficulty breathing, delayed growth or puberty, a painful erection in a male subject, and vision issues. Further, the administering the therapeutically effective amount of the RRx-001, or the pharmaceutically acceptable salt thereof, results in an amelioration of pain associated with the hemoglobinopathy in the subject such that a reduced amount of a pain-relieving agent (such as an opioid) is administered to the subject.

In some embodiments, the pain-relieving agent is the NSAID. In some embodiments, the pain-relieving agent is the opioid. In some embodiments, the pain-relieving agent is acetaminophen. In some embodiments, the pain-relieving agent is the anesthetic. In some embodiments, the pain-relieving agent is the sodium channel blocker. In some embodiments, the pain-relieving agent is the gabapentinoid (e.g., Neurontin or Gabapentin). In some embodiments, the pain-relieving agent is tramadol. In some embodiments, the pain-relieving agent is tapentadol. In some embodiments, the pain-relieving agent is the cannabinoid. In some embodiments, the pain-relieving agent is Ziconotide.

### Methods for Administering RRx-001

The therapeutically effective amount of the RRx-001, or the pharmaceutically acceptable salt thereof, may be administered to the subject through a variety of methods. In some embodiments, prior to administering the therapeutically effective amount of RRx-001, or the pharmaceutically acceptable salt thereof, the method comprises identifying the subject as being at risk for incurring the hemoglobinopathy or diagnosing the subject as suffering from the hemoglobinopathy. In some embodiments, the hemoglobinopathy is a sickle cell crisis.

In an exemplary embodiment, a subject's blood is withdrawn and extracorporeally combined with the therapeutically effective amount of the RRx-001, or the pharmaceutically acceptable salt thereof. The composition, which includes the therapeutically effective amount of the RRx-001, or the pharmaceutically acceptable salt thereof, and the blood product, is then re-introduced back to the subject.

In some embodiments, the blood product comprises erythrocyte cells. In some embodiments, the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis. In other embodiments, the blood product is a mixture of packed red blood cells. In some embodiments, the blood product is whole blood. In some embodiments, the whole blood is autologous whole blood. In some embodiments, the whole blood is donor-matched allogenic whole blood.

In some embodiments, the subject is a mammal subject. In some embodiments, the mammal subject is a human subject. In some embodiments, the mammal subject is a non-human subject.

In various embodiments, the therapeutically effective amount of the RRx-001, or the pharmaceutically acceptable salt thereof, is combined with one or more medicaments prior to administration to the subject. The one or more medicaments may comprise known compositions for treating sickle cell disease. In some embodiments, the therapeutically effective amount of the RRx-001, or the pharmaceutically acceptable salt thereof, is combined with hydroxyurea prior to administration to the subject.

The effective amount of RRx-001, or the pharmaceutically acceptable salt thereof, is a therapeutically effective amount of the RRx-001, or the pharmaceutically acceptable salt thereof. In various embodiments, the therapeutically effective amount of the RRx-001, or the pharmaceutically acceptable salt thereof, may be administered through a vehicle such as dimethyl sulfoxide (DMSO). In various embodiments, the therapeutically effective amount of the RRx-001, or the pharmaceutically acceptable salt thereof, is administered as an oral rinse, a drink to be consumed by the subject, an oral pill or tablet, an intravenous infusion, an intraperitoneal injection, a subcutaneous injection, through inhalation, or rectally.

In some embodiments, administering the therapeutically effective amount of RRx-001, or a pharmaceutically acceptable salt thereof, occurs via oral administration, transdermal administration, administration by inhalation, nasal administration, topical administration, intra-aural administration, rectal administration, intravenous administration, intramuscular administration, subcutaneous administration, or intraperitoneal administration, or combinations thereof. In some embodiments, administering the effective amount of RRx-001, or the pharmaceutically acceptable salt thereof, occurs via parenteral administration. In some embodiments, administering the therapeutically effective amount of RRx-001, or a pharmaceutically acceptable salt thereof, occurs via an oral administration. In some embodiments, administering the therapeutically effective amount of RRx-001, or a pharmaceutically acceptable salt thereof, occurs percutaneously. In some embodiments, administering the therapeutically effective amount of RRx-001, or a pharmaceutically acceptable salt thereof, occurs epicardially. In some embodiments, administering the therapeutically effective amount of RRx-001, or a pharmaceutically acceptable salt thereof, occurs endocardially. In some embodiments, administering the therapeutically effective amount of RRx-001, or a pharmaceutically acceptable salt thereof, occurs via an intravenous administration. In some embodiments, administering the therapeutically effective amount of RRx-001, or a pharmaceutically acceptable salt thereof, occurs via a direct intratumoral injection. In some embodiments, administering the effective amount of RRx-001, or the pharmaceutically acceptable salt thereof, occurs via intravenous administration, an oral administration, a subcutaneous administration, or an intramuscular administration.

In some embodiments, methods are provided to reduce or prevent vasoconstriction through the combination of RRx-001 with a nitric oxide-releasing or nitric oxide-prolonging compound, such as nitrite, gaseous nitric oxide, or a phosphodiesterase inhibitor. The nitric oxide-releasing or prolonging compound may be administered before, during, or after the administration of the effective amount of RRx-001, or the pharmaceutically acceptable salt thereof.

### Amount of RRx-001

The therapeutically effective amount of the RRx-001, or the pharmaceutically acceptable salt thereof, may be administered to the subject in various dosages at various frequencies and over various periods of time. Each of the dosage, frequency of the dosage, and the periods of time to which the dosage is administered may be adjusted based on factors, including, but not limited, to the medical condition of the subject, the desired outcome, and the one or more (if any) agents that are administered concurrently with the therapeutically effective amount of the RRx-001, or the pharmaceutically acceptable salt thereof.

In various embodiments, the dosage may be measured in units of a mass of the RRx-001 or the pharmaceutically acceptable salt thereof, a volume of the RRx-001 or the pharmaceutically acceptable salt thereof, a mass of the RRx-001 or the pharmaceutically acceptable salt thereof to a mass of the subject, a mass of the RRx-001 or the pharmaceutically acceptable salt thereof to a volume of the subject, a volume of the RRx-001 or the pharmaceutically acceptable salt thereof to a volume of the subject, a mass of the RRx-001 or the pharmaceutically acceptable salt thereof to a surface area of the subject, a volume of the RRx-001 or the pharmaceutically acceptable salt thereof to a surface area of the subject, or the like.

In some embodiments, the therapeutically effective amount of the RRx-001, or the pharmaceutically acceptable salt thereof, may be administered in one or more doses in a range of about 0.1 mg and about 500.0 mg. In some embodiments, the therapeutically effective amount of the RRx-001, or the pharmaceutically acceptable salt thereof, may be administered in one or more doses in a range of about 0.1 mg and about 200.0 mg. In some embodiments, the therapeutically effective amount of the RRx-001, or the pharmaceutically acceptable salt thereof, may be administered in one or more doses in a range of about 0.1 mg and about 0.5 mg, about 0.5 mg and about 1.0 mg, about 1.0 mg and about 1.5 mg, about 1.5 mg and about 2.0 mg, about 2.0 mg and about 2.5 mg, about 2.5 mg and about 3.0 mg, about 3.0 mg and about 3.5 mg, about 3.5 mg and about 4.0 mg, about 4.0 mg and about 4.5 mg, about 4.5 mg and about 5.0 mg, about 5.0 mg and about 6.0 mg, about 6.0 mg and about 7.0 mg, about 7.0 mg and about 8.0 mg, about 8.0 mg and about 9.0 mg, about 9.0 mg and about 10.0 mg, about 10.0 mg and about 15.0 mg, about 15.0 mg and about 20.0 mg, about 20.0 mg and about 25.0 mg, about 25.0 mg and about 30.0 mg, about 30.0 mg and about 35.0 mg, about 35.0 mg and about 40.0 mg, about 40.0 mg and about 45.0 mg, about 45.0 mg and about 50.0 mg, about 50.0 mg and about 55.0 mg, about 55.0 mg and about 60.0 mg, about 60.0 mg and about 65.0 mg, about 65.0 mg and about 70.0 mg, about 70.0 mg and about 75.0 mg, about 75.0 mg and about 80.0 mg, about 80.0 mg and about 85.0 mg, about 85.0 mg and about 90.0 mg, about 90.0 mg and about 95.0 mg, about 95.0 mg and about 100.0 mg, about 100.0 mg and about 110.0 mg, about 110.0 mg and about 120.0 mg, about 120.0 mg and about 130.0 mg, about 130.0 mg and about 140.0 mg, about 140.0 mg and about 150.0 mg, about 150.0 mg and about 160.0 mg, about 160.0 mg and about 170.0 mg, about 170.0 mg and about 180.0 mg, about 180.0 mg and about 190.0 mg, or about 190.0 mg and about 200.0 mg. In other embodiments, the therapeutically effective amount of the RRx-001, or the pharmaceutically acceptable salt thereof, may be administered in one or more doses in a range of about 100.0 mg and about 120.0 mg, about 120.0 mg and about 140.0 mg, about 140.0 mg and about 160.0 mg, about 160.0 mg and about 180.0 mg, about 200.0 mg and about 220.0 mg, about 220.0 mg and about 240.0 mg, about 240.0 mg and about 260.0 mg, about 260.0 mg and about 280.0 mg, about 280.0 mg and about 300.0 mg, about 300.0 mg and about 320.0 mg, about 320.0 mg and about 340.0 mg, about 340.0 mg and about 360.0 mg, about 360.0 mg and about 380.0 mg, about 380.0 mg and about 400.0 mg, about 400.0 mg and about 420.0 mg, about 420.0 mg and about 440.0 mg, about 440.0 mg and about 460.0 mg, about 460.0 mg and about 480.0 mg, or about 480.0 mg and about 500.0 mg.

### Frequency

Each dose of the therapeutically effective amount of the RRx-001, or the pharmaceutically acceptable salt thereof may be administered at various intervals over a time period until the end of a dosing period. The various intervals are referred to herein as a “frequency.”

In some embodiments, administration of the effective amount of RRx-001, or the pharmaceutically acceptable salt thereof, occurs via a single administration. In other embodiments, administration of the effective amount of RRx-001, or the pharmaceutically acceptable salt thereof, occurs via at least two administrations. In some embodiments, administration of the effective amount of RRx-001, or the pharmaceutically acceptable salt thereof, occurs at a frequency in a range of about 1 time per hour and about 1 time per month. In some embodiments, administration of the effective amount of RRx-001, or the pharmaceutically acceptable salt thereof, occurs at a frequency in a range of about 4 times per day and about 1 time per week. In some embodiments, administration of the effective amount of RRx-001, or the pharmaceutically acceptable salt thereof, occurs at a frequency in a range of about 2 times per day and about 3 times per week. In some embodiments, administration of the effective amount of RRx-001, or the pharmaceutically acceptable salt thereof, occurs at a frequency in a range of about one time a day. In some embodiments, administration of the effective amount of RRx-001, or the pharmaceutically acceptable salt thereof, occurs at a frequency in a range of about 1 time per day and about 2 times per week.

In some embodiments, each dose of the therapeutically effective amount of the RRx-001, or the pharmaceutically acceptable salt thereof, is administered at a frequency of about 1 dose per 24 hours. In another embodiment, each dose of the therapeutically effective amount of the RRx-001, or the pharmaceutically acceptable salt thereof, is administered at a frequency of about 1 dose per 48 hours. In some embodiments, each dose of the therapeutically effective amount of the RRx-001, or the pharmaceutically acceptable salt thereof, is administered at a frequency of about 3 doses per week. In another embodiment, each dose of the therapeutically effective amount of the RRx-001, or the pharmaceutically acceptable salt thereof, is administered at a frequency of about 2 doses per week. In some embodiments, each dose of the therapeutically effective amount of the RRx-001, or the pharmaceutically acceptable salt thereof, is administered at a frequency of about 1 dose per week. In other embodiments, each dose of the therapeutically effective amount of the RRx-001, or the pharmaceutically acceptable salt thereof, is administered at a frequency of about 2 doses per month. In some embodiments, each dose of the therapeutically effective amount of the RRx-001, or the pharmaceutically acceptable salt thereof, is administered at a frequency of about 1 dose per month. In other embodiments, each dose of the therapeutically effective amount of the RRx-001, or the pharmaceutically acceptable salt thereof, is administered at a frequency of about 1 dose per 2 months. In some embodiments, each dose of the therapeutically effective amount of the RRx-001, or the pharmaceutically acceptable salt thereof, is administered at a frequency of about 1 dose per 3 months.

In other embodiments, each dose of the therapeutically effective amount of the RRx-001, or the pharmaceutically acceptable salt thereof, is administered at a frequency of between about 1 dose per day and about 1 dose per week until the end of a dosing period. In some embodiments, each dose of the therapeutically effective amount of the RRx-001, or the pharmaceutically acceptable salt thereof, is administered at a frequency of between about 1 dose per day and about 2 doses per week until the end of a dosing period. In other embodiments, each dose of the therapeutically effective amount of the RRx-001, or the pharmaceutically acceptable salt thereof, is administered at a frequency of between about 1 dose per week and about 1 dose per month until the end of a dosing period. In other embodiments, each dose of the therapeutically effective amount of the RRx-001, or the pharmaceutically acceptable salt thereof, is administered at a frequency of between about 1 dose per month and about 1 dose per 3 months until the end of a dosing period.

### Pharmaceutical Composition

The present disclosure provides compositions or pharmaceutical compositions for treating a hemoglobinopathy and to reduce or prevent vasoconstriction in a subject in need thereof. As a general matter, the pharmaceutical composition contains at least one active agent and a pharmaceutically acceptable carrier. In some embodiments, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders a compound of the present invention orally bioavailable.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

In certain embodiments, it may be desirable to introduce the compositions disclosed herein into the central nervous system by any suitable route, including intraventricular, intrathecal and epidural injection. Intraventricular injection may be facilitated by an intraventricular catheter, for example, attached to a reservoir, such as an Ommaya reservoir.

In some embodiments, the pharmaceutical composition is configured as an inhalable formulation. In some embodiments, the inhalable formulation is configured as a dosage form adapted for pulmonary or nasal administration to the subject. In some embodiments, for example, dosage forms may include those adapted for inhalation such as aerosols and dry powders. In some embodiments, the formulation described herein is suitable for topical delivery to the lung via nose inhalation and/or mouth inhalation. In other embodiments, the compositions disclosed herein may also be administered directly to the lung by inhalation by a number of different devices.

In some embodiments, the inhalable formulation is configured as an aerosol formulation that comprises a propellant. In some embodiments, the propellant can provide energy to deliver molecules of any of the compounds described herein to the lung. Representative propellants are disclosed in U.S. Pat. No. 6,932,962 B1 and U.S. Pat. No. 8,367,734 B1, the contents of which are hereby incorporated by reference in their entirety. In some embodiments, the propellant is presented in the aerosol formulation in an amount ranging from 98% to 99% (w/w) relative to the total weight of the aerosol formulation.

In some embodiments, the aerosol formulation further comprises a surfactant, a co-solvent, and/or a pH buffer. The surfactant can give fine dispersions of the compounds described herein in the propellant and can stabilize the mixture of the compounds described herein in the propellant. In some embodiments, the surfactant comprises a fatty acid or a pharmaceutically acceptable salt thereof (e.g., a therapeutically effective amount), a bile salt, a phospholipid, or an alkyl saccharide. In some embodiments, the surfactant is presented in the formulations described herein in an amount of less than 5% (w/w) (e.g., less than 4%, less than 3%, less than 2%, less than 1% by weight) relative to the total weight of the aerosol formulation.

In some embodiments, the co-solvent can help to stabilize the surfactant and improve the dispersion characteristics. In some embodiments, exemplary co-solvents include ethyl alcohol, isopropyl alcohol, propylene glycol, ethylene glycol, propane, butane, isobutane, pentane, dimethyl ether, diethyl ether and the like. In some embodiments, the co-solvent is present in the formulation in an amount ranging from 0.5% to 20% w/w of the total weight of the formulation. In some embodiments, the co-solvent is present in the formulation in an amount ranging from 0.5% to 5% w/w of the total weight of the formulation. In some embodiments, the co-solvent is present in the formulation in an amount ranging from 0.5% to 1.5% (w/w) of the total weight of the formulation. Representative surfactants, co-solvents, and pH buffers are disclosed in U.S. Pat. No. 6,932,962 B1 and U.S. Pat. No. 8,367,734 B1, the contents of which are hereby incorporated by reference in their entirety.

In some embodiments, provided herein are combinations containing the aerosol formulation with the propellant and a pressurized bottle or a nebulizer. In some embodiments, the aerosol formulation with the propellant may be packed in pressurized bottles, where a dosage controller may be used with the pressurized bottle to control the amount of drug being administrated in each spray. In some embodiments, the aerosol formulation with the propellant may be packed in pressurized bottles with a dosage controller, where the dosage controller comprises a valve that controls the delivery of a metered amount of the drug.

In some embodiments, the aerosol formulation is propellant-free and comprises the effective amount of the RRx-001 or the pharmaceutical composition and a solvent. In some embodiments, exemplary solvents include water and alcohols, such as ethanol, isopropanol, and glycols, such as propylene glycol, polyethylene glycol, polypropylene glycol, glycol ether, glycerol and polyoxyethylene alcohols. In some embodiments, the solvent is present in the propellant-free aerosol formulation in an amount ranging from about 0.01% to about 90% (w/w), or about 0.01% to about 50% (w/w), or about 0.01% to about 25% (w/w), or about 0.01% to about 10% (w/w), or about 0.01% to about 5% (w/w) relative to the total weight of the aerosol formulation.

In some embodiments, the propellant-free aerosol formulation may further comprise an emulsifying agent. In some embodiments, exemplary emulsifying agents are disclosed in U.S. Pat. No. 9,498,437 B2, the contents of which are hereby incorporated by reference in its entirety. In some embodiments, the emulsifying agent is present in the propellant-free aerosol formulations in an amount ranging from about 0.001% to about 50% (w/w), or about 0.001% to about 25% (w/w), or about 0.001% to about 10% (w/w), or about 0.001% to about 2% (w/w), or about 0.001% to about 1% (w/w) relative to the total weight of the aerosol formulation.

In some embodiments, the propellant-free aerosol formulation may further comprise a complexing agent. In some embodiments, exemplary complexing agents include, but are not limited to, ethylenediaminetetraacetic acid (EDTA) or a pharmaceutically acceptable salt thereof (e.g., a therapeutically effective amount), such as the disodium salt, citric acid, nitrilotriacetic acid and the salts thereof, and sodium edetate. Representative complexing agents are disclosed in U.S. Pat. No. 9,498,437 B2, the contents of which are hereby incorporated by reference in its entirety. In some embodiments, the complexing agent is present in the propellant-free aerosol formulations in an amount ranging from about 0.001% to about 50% (w/w), or about 0.001% to about 25% (w/w), or about 0.001% to about 10% (w/w), or about 0.001% to about 2% (w/w), or about 0.001% to about 1% (w/w) relative to the total weight of the aerosol formulation.

In some embodiments, the propellant-free aerosol formulation may further comprise a tonicity agent that can adjust the isotonicity of the present formulations. In some embodiments, exemplary tonicity agents include, but are not limited to, sodium chloride, potassium chloride, zinc chloride, calcium chloride or mixtures thereof. Other osmotic adjusting agents may also include, but are not limited to, mannitol, glycerol, and dextrose or mixtures thereof.

Representative tonicity agents are disclosed in U.S. Pat. No. 9,498,437 B2, the contents of which are hereby incorporated by reference in its entirety. In some embodiments, the tonicity agent is present in the propellant-free aerosol formulations in an amount ranging from about 0.01% to about 10% (w/w), or about 1% to about 10% (w/w), or about 1% to about 6% (w/w) relative to the total weight of the aerosol formulation. In some embodiments, the aerosol formulation may further comprise the pH buffer.

In some embodiments, provided herein are combinations containing the propellent-free aerosol formulation provided herein and a nebulizer. In some embodiments, the nebulizer can nebulize liquid formulations, including the propellant-free aerosol formulations detailed herein, and produce a nebulized aerosol mist. In some embodiments, the nebulizer may further have an internal baffle, which can selectively remove large droplets from the mist by impaction and allow the droplets to return to the reservoir, so that only fine aerosol droplets are entrained into the lung of the subject by the inhaling air/oxygen. Examples of nebulizers include devices supplied by Sheffield Pharmaceuticals, St. Louis, MO. (Armer et al., U.S. Pat. No. 5,954,047; van der Linden et al., U.S. Pat. No. 5,950,619; van der Linden et al., U.S. Pat. No. 5,970,974, the contents of which are hereby incorporated by reference in their entirety) and Batelle Pulmonary Therapeutics, Columbus, OH).

In some embodiments, a Metered Dose Inhaler (“MDI”), which utilizes canisters that contain a suitable low boiling propellant, (e.g., dichlorodifluoromethane, trichlorofluoromethane, dichlorotetrafluoroethane, carbon dioxide or any other suitable gas) may be used to deliver the RRx-001 and/or pharmaceutical compositions thereof directly to the lung. Specifically, the MDI comprises an aerosol container suitable for containing a propellant-based aerosol formulation and/or a metering valve, for example a side valve, which controls the release of the aerosol formulation to the subject. Representative methods and devices to administer the aerosol formulation with the propellant are disclosed in U.S. Pat. No. 9,498,437 B2, the contents of which are hereby incorporated by reference in its entirety.

In another embodiment, a Dry Powder Inhaler (“DPI”) device may be used to administer the compositions disclosed herein to the lung. DPI devices typically use a mechanism such as a burst of gas to create a cloud of dry powder inside a container, which may then be inhaled by the patient and are well known in the art. In a particular embodiment, a popular variation is the multiple dose DPI (“MDDPI”) system, which allows for the delivery of more than one therapeutic dose. MDDPI devices are commercially available from a number of pharmaceutical companies e.g., Schering Plough, Madison, NJ). For example, capsules and cartridges of gelatin for use in an inhaler or insufflator may be formulated containing a powder mix of the compositions disclosed herein and a suitable powder base such as lactose or starch for these systems.

In some embodiments, another type of device that may be used to deliver the compositions disclosed herein to the lung is a liquid spray device supplied, for example, by Aradigm Corporation, Hayward, CA. Liquid spray systems use extremely small nozzle holes to aerosolize liquid drug formulations that may then be directly inhaled into the lung.

In some embodiments, a nebulizer is used to deliver the compositions disclosed herein to the lung. Nebulizers create aerosols from liquid drug formulations by using, for example, ultrasonic energy to form fine particles that may be readily inhaled (see e.g., Verschoyle et al., British J. Cancer, 1999, 80, Suppl. 2, 96, the contents of which are hereby incorporated by reference in its entirety). Examples of nebulizers include devices supplied by Sheffield Pharmaceuticals, St. Louis, MO. (Armer et al., U.S. Pat. No. 5,954,047; van der Linden et al., U.S. Pat. No. 5,950,619; van der Linden et al., U.S. Pat. No. 5,970,974) and Batelle Pulmonary Therapeutics, Columbus, OH).

In other embodiments, an electrohydrodynamic (“EHD”) aerosol device is used to deliver the compositions disclosed herein to the lung of a patient. EHD aerosol devices use electrical energy to aerosolize liquid drug solutions or suspensions (see e.g., Noakes et al., U.S. Pat. No. 4,765,539, the contents of which are hereby incorporated by reference in its entirety). The electrochemical properties of the formulation may be important parameters to optimize when delivering the RRx-001 and/or pharmaceutical composition thereof to the lung with an EHD aerosol device. EHD aerosol devices may more efficiently deliver drugs to the lung than existing pulmonary delivery technologies.

Suitable pharmaceutically acceptable excipients will vary depending upon the particular dosage form chosen. In some embodiments, for example, certain pharmaceutically acceptable excipients may be chosen for their ability to: facilitate the production of aerosol for inhalation, facilitate the production of solution or mist for inhalation, facilitate the production of dry powder for inhalation, or facilitate the production of stable dosage forms.

In some embodiments, the compositions disclosed herein can be delivered via sustained release systems, e.g., oral sustained release systems. In other embodiments, a pump may be used (e.g., Langer, supra, Sefton, 1987, CRC Crit. Ref Biomed. Eng. 14:201; Saudek et al., 1989, N. Engl. J Med. 321:574, the contents of which are hereby incorporated by reference in their entirety).

In some embodiments, polymeric materials can be used (e.g., “Medical Applications of Controlled Release,” Langer and Wise (eds.), CRC Press, Boca Raton, Florida (1974); “Controlled Drug Bioavailability,” Drug Product Design and Performance, Smolen and Ball (eds.), Wiley, New York (1984); Ranger et al., 1983, J Macromol. Sci. Rev. Macromol Chem. 23:61; Levy et al., 1985, Science 228:190; During et al., 1989, Ann. Neurol. 25:351; Howard et al., 1989, J. Neurosurg. 71:105, the contents of which are hereby incorporated by reference in their entirety).

In other embodiments, polymeric materials are used for oral sustained release delivery. Polymers include, but are not limited to, sodium carboxymethylcellulose, hydroxypropylcellulose, hydroxypropylmethylcellulose and hydroxyethylcellulose (most preferred, hydroxypropyl methylcellulose). Other cellulose ethers have been described (Alderman, Int. J. Pharm. Tech. & Prod. Mfr. 1984, 5 (3) 1-9, the contents of which are hereby incorporated by reference in its entirety). Factors affecting drug release are well known to the skilled artisan and have been described in the art (Bamba et al., Int. J. Pharm. 1979, 2, 307, the contents of which are hereby incorporated by reference in its entirety).

In other embodiments, enteric-coated preparations can be used for oral sustained release administration. Coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In other embodiments, osmotic delivery systems are used for oral sustained release administration (Verma et al., Drug Dev. Ind. Pharm., 2000, 26:695-708, the contents of which are hereby incorporated by reference in its entirety). In some embodiments, OROS™ osmotic devices are used for oral sustained release delivery devices (Theeuwes et al., U.S. Pat. No. 3,845,770; Theeuwes et al., U.S. Pat. No. 3,916,899, the contents of which are hereby incorporated by reference in their entirety).

In yet other embodiments, a controlled-release system can be placed in proximity of the target of RRx-001 described herein and/or pharmaceutical composition, thus requiring only a fraction of the systemic dose (e.g., Goodson, in “Medical Applications of Controlled Release,” supra, vol. 2, pp. 115-138 (1984), the contents of which are hereby incorporated by reference in its entirety). Other controlled-release systems previously may also be used (Langer, 1990, Science 249:1527-1533, the contents of which are hereby incorporated by reference in its entirety).

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug administered by subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to human and animal subjects, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Parenteral administrations are preferred.

Combination with a Blood Product and/or Another Agent

The pharmaceutical composition comprises the effective amount of RRx-001, or a pharmaceutically acceptable salt thereof (e.g., a therapeutically effective amount), and at least one pharmaceutically acceptable carrier or excipient. In some embodiments, the pharmaceutical composition comprises (1) an effective amount of RRx-001, or a pharmaceutically acceptable salt thereof and (2) at least one of a blood product and an additional agent. In various embodiments disclosed herein, the composition comprising the effective amount of RRx-001, or a pharmaceutically acceptable salt thereof, is combined with a blood product prior to administration to the subject. The additional agent may comprise hydroxyurea. Depending on the circumstances, the pharmaceutical composition is pre-mixed with a sample of blood harvested from the subject prior to administration. Methods of performing blood transfusion by administering to a subject in need thereof are described in WO2017/123593, the contents of which are hereby incorporated by reference in its entirety.

The present invention can provide methods of attenuating interactions of a first drug (e.g., a first therapeutic agent) and a second drug (e.g., a second therapeutic agent) in a mammal. As described herein, interactions of drugs, or drug-drug interactions, can refer to the changes of the effects of a drug or a pharmaceutical composition on a mammal when the pharmaceutical composition is taken together with a second drug or second pharmaceutical composition. In some embodiments, the interactions can occur when more than two drugs are concurrently in a mammal, regardless of the time between the administrations of the two or more drugs and thereby, and react with each other.

In some embodiments, an attenuating interaction of drugs includes actions that result in reducing or preventing any types of interactions between two or more drugs or reducing the hypersensitivity, the toxicity, or adverse effects that are caused by the interactions of two or more drugs. In some embodiments, the interactions can include, but are not limited to, synergistic or antagonistic interactions. By way of examples, attenuating interactions of the drugs can be at least any one of the following scenarios: reducing and/or preventing drug-drug physical interactions, reducing and/or preventing drug-drug pharmacokinetic interactions, reducing and/or preventing the hypersensitivity caused by co-existence of the drugs, reducing and/or preventing the toxicity caused by co-existence of drugs, or reducing and/or preventing the antagonistic interactions of drugs.

In some embodiments, the effects of the attenuated interactions can be delayed, decreased, or enhanced absorption of either pharmaceutical composition, and thereby decreases or increases the action of one or more of the additional agent(s) or the pharmaceutical composition. In some embodiments, the attenuated interactions can impact the transport or the distribution of the additional agent(s) or the pharmaceutical compositions.

Accordingly, in certain embodiments, the subject has reduced incidence and/or severity of side effects compared to subjects receiving a direct administration of the same additional agent(s) at the same dose without being mixed with the blood product prior to administration. In certain embodiments, the subject has reduced side effects compared to subjects receiving a direct administration of the same additional agent(s) at the same dose without being mixed with the blood product prior to administration. In certain embodiments, the dose of the additional agent(s) in the pharmaceutical composition is at least about 10% to about 300% more than the dose recommended for a direct administration of the same additional agent(s) without being mixed with the blood product prior to administration. In certain embodiments, the dose of the additional agent(s) in the pharmaceutical composition is at least 1%, at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, or higher, inclusive of all ranges and subranges therebetween, more than the dose recommended for a direct administration of the same additional agent(s) without being mixed with the blood product prior to administration.

In certain embodiments, the additional agent(s) has/have a longer circulating half-life in the subject compared to direct administration of the same additional agent(s) at the same dose without being mixed with the blood product prior to administration. In certain embodiments, the circulating half-life of the additional agent(s) is at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 110%, 110%, 120%, 130%, 140%, 150%, 160%, 170%, 180%, 190%, 200%, 250%, 300%, 350%, 400%, 450%, 500%, 600%, 700%, 800%, 900%, 1000%, or more, longer than the circulating half-life of the same additional agent(s) at the same dose without being mixed with the blood product before administration.

### Example

In order that this disclosure may be more fully understood, the following Example is set forth. It should be understood that this example is for illustrative purposes only and is not to be construed as limiting this disclosure in any manner.

### Example 1. Assessing the Efficacy of RRx-001 as a Treatment for Sickle Cell Disease

This example was conducted on Townes-SS mice (or subjects) to measure an effect of RRx-001 on the subjects to ameliorate symptoms of sickle cell disease. Townes-SS mice contain two human sickle Hb alleles and exhibit hematologic sickle-shaped erythrocytes, increased reticulocytes, and anemia. The Townes-SS mice were implanted with dorsal skinfold window chambers to allow for continuous observation of the vasculature in the mice. The instrumented Townes-SS mice were treated with three doses of RRx-001 (5 mg/kg, IP every other day). Next, the Townes-SS mice were anesthetized and a carotid catheter as implanted, exteriorized, and secured to the chamber. Finally, two days after catheter implantation, the Townes-SS mice were restrained in an acrylic tube, and the window chamber was studied via intravital microscopy.

About 12 to 14 flowing arterioles and 18 to 26 venules, and 12 capillary fields were selected and measured. Parameters for measurement included diameter and red blood cell velocity and FCD. After baseline measurements, the Townes-SS mice were challenged with hypoxia-reoxygenation whereby the Townes-SS mice were exposed to an atmosphere of 7% oxygen and 93% nitrogen for 1 hour followed by reoxygenation with room air. Various parameters were measured, such as blood pressure and heart rate, blood gas parameters, hematological parameters, echocardiography and cerebral flow, microvascular blood flow, FCD, and red blood cell leukocyte adhesion.

FIG. 1A is a graph 100 depicting hemoglobin oxygen saturation (HbO₂ Sat) as compared to oxygen pressure (PO₂, mmHg) to measure oxygen affinity. The graph 100 displays PO₂ on an x-axis and HbO₂ Sat on a y-axis. Data for a vehicle (control group) and data for the subject group treated with RRx-001 weekly for three weeks with 5 mg/kg RRx-001 is plotted for oxygen pressures from 0 mmHg to 150 mmHg on the x-axis.

FIG. 1B is a graph 150 of hemoglobin polymerization as measured by turbidity (A_{700 nm}) over time (measured in minutes). Specifically, the x-axis depicts time and the y-axis depicts turbidity, which is measured by absorption at 700 nm. Turbidity measurements were plotted for vehicle (control) and RRx-001 subject groups from a time period of 0 minutes to 400 minutes. The subjects that were administered RRx-001 showed significantly less turbidity as compared to the subjects in the vehicle group over most of the time.

FIG. 2 is a graph 200 showing changes in various cytoprotectant levels subsequent to administration of RRx-001. Specifically, the x-axis depicts the measured cytoprotectant levels including: HMOX, ferritin heavy chain, Hp, hemopexin, ferroportin, GCL, glutathione transferase alpha, NAD (P) dehydrogenase, and glutathione S-transferase η1. Measurements from the subjects in the RRx-001 group were shaded and measurements from the subjects in the vehicle (control) group were unshaded. The graph 200 shows that measurements for all cytoprotectant levels in the RRx-001 treated subjects were higher than the relative cytoprotectant measurements for the subjects in the vehicle (control) group.

FIG. 3A is a graph 300 showing a change in bilirubin concentration for a vehicle (control) group and for a subject group treated with RRx-001. The graph 300 shows that the average serum bilirubin concentration in mg/dL for the subjects in the RRx-001 group was about 5 mg/dL. Further, the graph 300 shows that the average serum bilirubin concentration for the subjects in the vehicle (control) group was about 8 mg/dL.

FIG. 3B is a graph 320 showing a change in ferritin concentration for a vehicle (control) group and for a subject group treated with RRx-001. As shown in the graph 320, the average ferritin concentration for the subject mice in the RRx-001 group was approximately 6200 μg/L. The average ferritin concentration for subject mice in the vehicle (control) group was approximately 3700 μg/L.

FIG. 3C is a graph 340 showing a change in transferrin saturation percentage for a vehicle (control) group and for a subject group treated with RRx-001. As shown in the graph 340, the average saturation percentage of transferrin in subject mice in the RRx-001 group was approximately 37%. The average saturation percentage of transferrin in subject mice in the RRx-001 vehicle (control) group was approximately 56%.

FIG. 4A is a photograph 400 of a dorsal skinfold window chamber as it displays a vasculature of a Townes-SS mouse. FIG. 4B is a magnified photograph 450 of a dorsal skinfold window chamber as it displays a vasculature of a Townes mouse. The dorsal skinfold window chamber allows for continuous observation of a vasculature through the window.

FIG. 5A is a graph 1000 showing the number of small venules in groups of subject mice over a three-day period. The average number of small venules for the subject mice in the HbAA-Townes group was approximately 29 for baseline, 28 at 0 hours, 28 at 24 hours, 30 at 48 hours, and 29 at 72 hours. The average number of small venules for the subject mice in the vehicle group was approximately 28 for baseline, 19 at 0 hours, 13 at 24 hours, 13 at 48 hours, and 19 at 72 hours. The average number of small venules for the subject mice in the RRx-001 group was approximately 29 for baseline, 20 at 0 hours, 17 at 24 hours, 20 at 48 hours, and 27 at 72 hours. The average number of small venules for the subject mice in the RRx-001 group was greater than the average number of small venules for the subject mice in the HbAA-Townes group and the average number of small venules for the subject mice in the vehicle group at 72 hours.

FIG. 5B is a graph 1020 showing the number of small venules in groups of subject mice over a three-day period normalized to a baseline (BL) measurement. The average normalized number of small venules for the subject mice in the HbAA-Townes group was approximately 0.9 at 0 hours, 0.9 at 24 hours, 1.0 at 48 hours, and 1.0 at 72 hours. The average normalized number of small venules for the subject mice in the vehicle group was approximately 0.7 at 0 hours, 0.5 at 24 hours, 0.5 at 48 hours, and 0.7 at 72 hours. The average normalized number of small venules for the subject mice in the RRx-001 group was approximately 0.7 at 0 hours, 0.6 at 24 hours, 0.7 at 48 hours, and 0.9 at 72 hours. The average normalized number of small venules for the subject mice in the RRx-001 group was greater than the average normalized number of small venules for the subject mice in the HbAA-Townes group and the average normalized number of small venules for the subject mice in the vehicle group at 72 hours.

FIG. 6A is a graph 1100 of FCD in vessels per unit area in groups of subject mice over a three-day period. The average FCD in vessels per area for the subject mice in the HbAA-Townes group was approximately 67 for baseline, 61 at 0 hours, 61 at 24 hours, 64 at 48 hours, and 70 at 72 hours. The average FCD in vessels per area for the subject mice in the vehicle group was approximately 78 for baseline, 40 at 0 hours, 18 at 24 hours, 17 at 48 hours, and 20 at 72 hours. The average FCD in vessels per area for the subject mice in the RRx-001 group was approximately 75 for baseline, 49 at 0 hours, 35 at 24 hours, 48 at 48 hours, and 55 at 72 hours.

FIG. 6B is a graph 1120 of FCD normalized to a baseline (BL) measurement in groups of subject mice over a three-day period. The average normalized FCD for the subject mice in the HbAA-Townes group was approximately 0.9 at 0 hours, 1.0 at 24 hours, 1.0 at 48 hours, and 1.0 at 72 hours. The average normalized FCD for the subject mice in the vehicle group was approximately 0.5 at 0 hours, 0.2 at 24 hours, 0.2 at 48 hours, and 0.3 at 72 hours. The average normalized FCD for the subject mice in the RRx-001 group was approximately 0.6 at 0 hours, 0.5 at 24 hours, 0.6 at 48 hours, and 0.7 at 72 hours.

### OTHER EMBODIMENTS

This application refers to various published patent applications, journal articles, and other publications, each of which is incorporated herein by reference.

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method for treating a hemoglobinopathy and to reduce or prevent vasoconstriction in a subject, the method comprising administering to the subject in need thereof an effective amount of RRx-001, or a pharmaceutically acceptable salt thereof.

2. The method of claim 1, wherein the effective amount of RRx-001, or the pharmaceutically acceptable salt thereof, is a therapeutically effective amount of the RRx-001, or the pharmaceutically acceptable salt thereof.

3. The method of claim 2, wherein the therapeutically effective amount of the RRx-001, or the pharmaceutically acceptable salt thereof, is in a range of about 0.1 mg and about 500.0 mg.

4. The method of claim 3, wherein the therapeutically effective amount of the RRx-001, or the pharmaceutically acceptable salt thereof, is in a range of about 0.5 mg and about 200.0 mg.

5. The method of any of claims 2-4, wherein the therapeutically effective amount of the RRx-001, or the pharmaceutically acceptable salt thereof, results in an increase in at least one of: nitric oxide (NO), heme oxygenase (HMOX), ferritin heavy chain, haptoglobin (Hp), hemopexin, ferroportin, glutamate-cysteine ligase (GCL), glutathione transferase alpha, NAD (P) dehydrogenase, glutathione S-transferase n1, a concentration of ferritin, capillary density, oxygen delivery, oxygen uptake, a quantity of flowing venules, and functional capillary density in the subject.

6. The method of any of claims 2-5, wherein the therapeutically effective amount of the RRx-001, or the pharmaceutically acceptable salt thereof, results in a decrease in at least one of: a concentration of bilirubin, a heart rate, and a lactate concentration in the subject.

7. The method of any of claims 2-6, wherein the therapeutically effective amount of the RRx-001, or the pharmaceutically acceptable salt thereof, results in an amelioration of at least one adverse event of the hemoglobinopathy in the subject.

8. The method of claim 7, wherein each adverse event of the at least one adverse event is a vaso-occlusive crisis (VOC).

9. The method of claim 7, wherein each adverse event of the at least one adverse event is selected from the group consisting of: anemia, pain or swelling in at least one extremity, chest pain, lower back pain, a headache, tiredness, jaundice, difficulty breathing, delayed growth or puberty, painful erection in a male subject, and vision issues.

10. The method of any of claims 2-9, wherein the therapeutically effective amount of the RRx-001, or the pharmaceutically acceptable salt thereof, results in an amelioration of pain associated with the hemoglobinopathy in the subject such that a reduced amount of a pain-relieving agent is administered to the subject.

11. The method of claim 10, wherein the pain-relieving agent is selected from the group consisting of: a nonsteroidal anti-inflammatory agent (NSAID), an opioid, an anesthetic, a sodium channel blocker, a gabapentinoid, and a cannabinoid.

12. The method of any of claims 1-11, wherein, prior to administering the effective amount of RRx-001, or the pharmaceutically acceptable salt thereof, the method further comprises identifying the subject as being at risk for incurring the hemoglobinopathy.

13. The method of any of claims 1-11, wherein, prior to administering the effective amount of RRx-001, or the pharmaceutically acceptable salt thereof, the method further comprises diagnosing the subject as suffering from the hemoglobinopathy.

14. The method of any one of claims 12-13, wherein the hemoglobinopathy is a sickle cell crisis.

15. The method of any of claims 2-14, wherein administering the therapeutically effective amount of RRx-001, or the pharmaceutically acceptable salt thereof, is performed via a single administration.

16. The method of any of claims 2-14, wherein administering the therapeutically effective amount of RRx-001, or the pharmaceutically acceptable salt thereof, is performed via at least two administrations.

17. The method of claim 16, wherein administering the therapeutically effective amount of RRx-001, or the pharmaceutically acceptable salt thereof, is performed a frequency in a range of about 1 time per hour and about 1 time per month.

18. The method of claim 16, wherein administering the therapeutically effective amount of RRx-001, or a pharmaceutically acceptable salt thereof, is performed at a frequency of between about 4 times per day and about 1 time per week.

19. The method of claim 16, wherein administering the therapeutically effective amount of RRx-001, or a pharmaceutically acceptable salt thereof, is performed at a frequency of between about 2 times per day and about 3 times per week.

20. The method of claim 16, wherein administering the therapeutically effective amount of RRx-001, or a pharmaceutically acceptable salt thereof, is performed at a frequency of about one time a day.

21. The method of claim 16, wherein administering the therapeutically effective amount of RRx-001, or a pharmaceutically acceptable salt thereof, is performed at a frequency of between about 1 time per day and about 2 times per week.

22. The method of any of claims 1-21, wherein the subject is a mammal subject.

23. The method of claim 22, wherein the mammal subject is a human subject.

24. The method of claim 22, wherein the mammal subject is a non-human subject.

25. The method of any one of claims 2-24, wherein administering the therapeutically effective amount of RRx-001, or a pharmaceutically acceptable salt thereof, occurs via oral administration, transdermal administration, administration by inhalation, nasal administration, topical administration, intra-aural administration, rectal administration, intravenous administration, intramuscular administration, subcutaneous administration, or intraperitoneal administration, or combinations thereof.

26. The method of any of claims 2-24, wherein administering the therapeutically effective amount of RRx-001, or the pharmaceutically acceptable salt thereof, occurs via a parenteral administration.

27. The method of any one of claims 2-24, wherein administering the therapeutically effective amount of RRx-001, or a pharmaceutically acceptable salt thereof, occurs via an oral administration.

28. The method of any one of claims 2-24, wherein administering the therapeutically effective amount of RRx-001, or a pharmaceutically acceptable salt thereof, occurs percutaneously.

29. The method of any one of claims 2-24, wherein administering the therapeutically effective amount of RRx-001, or a pharmaceutically acceptable salt thereof, occurs epicardially.

30. The method of any one of claims 2-24, wherein administering the therapeutically effective amount of RRx-001, or a pharmaceutically acceptable salt thereof, occurs endocardially.

31. The method of any one of claims 2-24, wherein administering the therapeutically effective amount of RRx-001, or a pharmaceutically acceptable salt thereof, occurs via an intravenous administration.

32. The method of any one of claims 2-24, wherein administering the therapeutically effective amount of RRx-001, or a pharmaceutically acceptable salt thereof, occurs via a direct intratumoral injection.

33. The method of any of claims 2-32, further comprising: administering an agent prior to, concurrently with, or subsequent administering the therapeutically effective amount of RRx-001, or a pharmaceutically acceptable salt thereof.

34. The method of claim 33, wherein the agent comprises a nitric oxide-releasing or prolonging compound.

35. The method of claim 34, wherein the nitric oxide-releasing or prolonging compound is selected from the group consisting of: a phosphodiesterase inhibitor, inhaled nitric oxide, inhaled carbon monoxide, and nitrite.

36. A pharmaceutical composition for treating a hemoglobinopathy and to reduce or prevent vasoconstriction in a subject in need thereof, the pharmaceutical composition comprising an effective amount of RRx-001, or a pharmaceutically acceptable salt thereof.

37. The pharmaceutical composition of claim 36, wherein the effective amount of RRx-001, or the pharmaceutically acceptable salt thereof, is a therapeutically effective amount of the RRx-001, or the pharmaceutically acceptable salt thereof.

38. The pharmaceutical composition of claim 37, wherein the therapeutically effective amount of the RRx-001, or the pharmaceutically acceptable salt thereof, is in a range of about 0.1 mg and about 500.0 mg.

39. The pharmaceutical composition of claim 38, wherein the therapeutically effective amount of the RRx-001, or the pharmaceutically acceptable salt thereof, is in a range of about 0.5 mg and about 200.0 mg.

40. The pharmaceutical composition of any one of claims 36-39, wherein, prior to administering the pharmaceutical composition, the method further comprises identifying the subject as being at risk for incurring the hemoglobinopathy.

41. The pharmaceutical composition of any one of claims 36-40, wherein, prior to administering the pharmaceutical composition, the method further comprises diagnosing the subject as suffering from the hemoglobinopathy.

42. The pharmaceutical composition of any one of claims 40-41, wherein the hemoglobinopathy is a sickle cell crisis.

43. The pharmaceutical composition of any one of claims 36-42, wherein the pharmaceutical composition further comprises a blood product.

44. The pharmaceutical composition of claim 43, wherein the blood product comprises erythrocyte cells.

45. The pharmaceutical composition of claim 44, wherein the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis.

46. The pharmaceutical composition of any one of claims 43-45, wherein the blood product is a mixture of packed red blood cells.

47. The pharmaceutical composition of any one of claims 43-45, wherein the blood product is whole blood.

48. The pharmaceutical composition of claim 47, wherein the whole blood is autologous whole blood.

49. The pharmaceutical composition of any one of claims 36-48, wherein the pharmaceutical composition further comprises hydroxyurea.

50. The pharmaceutical composition of any one of claims 37-49, wherein administering the pharmaceutical composition occurs via a single administration.

51. The pharmaceutical composition of any one of claims 37-49, wherein administering the pharmaceutical composition occurs via at least two administrations.

52. The pharmaceutical composition of claim 51, wherein administering the pharmaceutical composition occurs at a frequency in a range of about 1 time per hour and about 1 time per month.

53. The pharmaceutical composition of claim 51, wherein administering the pharmaceutical composition occurs at a frequency between about 4 times per day and about 1 time per week.

54. The pharmaceutical composition of claim 51, wherein administering the pharmaceutical composition occurs at a frequency between about 2 times per day and about 3 times per week.

55. The pharmaceutical composition of claim 51, wherein administering the pharmaceutical composition occurs at a frequency of about one time a day.

56. The pharmaceutical composition of claim 51, wherein administering the pharmaceutical composition occurs at a frequency between about 1 time per day and about 2 times per week.

57. The pharmaceutical composition of any one of claims 36-56, wherein the subject is a mammal subject.

58. The pharmaceutical composition of claim 57, wherein the mammal subject is a human subject.

59. The pharmaceutical composition of claim 57, wherein the mammal subject is a non-human subject.

60. The pharmaceutical composition of any one of claims 37-59, wherein administering the pharmaceutical composition occurs via oral administration, transdermal administration, administration by inhalation, nasal administration, topical administration, intra-aural administration, rectal administration, intravenous administration, intramuscular administration, subcutaneous administration, or intraperitoneal administration, or combinations thereof.

61. The pharmaceutical composition of any one of claims 37-59, wherein administering the pharmaceutical composition occurs via a parenteral administration.

62. The pharmaceutical composition of any one of claims 37-59, wherein administering the pharmaceutical composition occurs via an oral administration.

63. The pharmaceutical composition of any one of claims 37-59, wherein administering the pharmaceutical composition occurs percutaneously.

64. The pharmaceutical composition of any one of claims 37-59, wherein administering the pharmaceutical composition occurs epicardially.

65. The pharmaceutical composition of any one of claims 37-59, wherein administering the pharmaceutical composition occurs endocardially.

66. The pharmaceutical composition of any one of claims 37-59, wherein administering the pharmaceutical composition occurs via an intravenous administration.

67. The pharmaceutical composition of any one of claims 37-59, wherein administering the pharmaceutical composition occurs via a direct intratumoral injection.

68. The pharmaceutical composition of any one of claims 37-67, wherein administering the pharmaceutical composition results in an increase in at least one of: nitric oxide (NO), heme oxygenase (HMOX), ferritin heavy chain, haptoglobin (Hp), hemopexin, ferroportin, glutamate-cysteine ligase (GCL), glutathione transferase alpha, NAD (P) dehydrogenase, glutathione S-transferase η1, a concentration of ferritin, capillary density, oxygen delivery, oxygen uptake, a quantity of flowing venules, and functional capillary density in the subject.

69. The pharmaceutical composition of any one of claims 37-68, wherein administering the pharmaceutical composition results in a decrease in at least one of: a concentration of bilirubin, a heart rate, and a lactate concentration in the subject.

70. The pharmaceutical composition of any one of claims 37-69, wherein administering the pharmaceutical composition results in results in an amelioration of at least one adverse event of the hemoglobinopathy in the subject.

71. The pharmaceutical composition of claim 70, wherein each adverse event of the at least one adverse event is a vaso-occlusive crisis (VOC).

72. The pharmaceutical composition of claim 71, wherein each adverse event of the at least one adverse event is selected from the group consisting of: anemia, pain or swelling in at least one extremity, chest pain, lower back pain, a headache, tiredness, jaundice, difficulty breathing, delayed growth or puberty, painful erection in a male subject, and vision issues.

73. The pharmaceutical composition of any one of claims 37-72, wherein administering the pharmaceutical composition results in results in an amelioration of pain associated with the hemoglobinopathy in the subject such that a reduced amount of a pain-relieving agent is administered to the subject.

74. The pharmaceutical composition of claim 72, wherein the pain-relieving agent is selected from the group consisting of: a nonsteroidal anti-inflammatory agent (NSAID), an opioid, an anesthetic, a sodium channel blocker, a gabapentinoid, and a cannabinoid.
